(19) United States
(12) Patent Application Publication
(10) Pub. No.: US 2002/0128231 A1
(43) Pub. Date: Sep. 12, 2002

(54) Aryl-indane compounds

(76) Inventor: Anita Melikian-Badalian, San Francisco, CA (US)

Correspondence Address: COOLEY GODWARD, LLP, 3000 EL CAMINO REAL, 5 PALO ALTO SQUARE, PALO ALTO, CA, 94306, US

(21) Appl. No.: 09/976,929

(22) Filed: Oct. 11, 2001

Related U.S. Application Data
(60) Provisional application No. 60/240,345, filed on Oct. 11, 2000.

(51) Int. Cl. A61K 31/695; A61K 31/137; A61K 31/357; A61K 31/195

(52) U.S. Cl. 514/63; 514/452; 514/463; 514/532; 514/534; 514/567; 514/618; 514/647; 514/649

(57) ABSTRACT

A new family of compounds, particularly aryl-indane compounds, is disclosed. The compounds may be used as inhibitors of P-glycoprotein-mediated transport. Use of the compounds to enhance bioavailability and to modulate multi-drug resistance to chemotherapeutic agents is disclosed.

### CROSS REFERENCE TO RELATED APPLICATIONS

This patent application claims priority to U.S. Provisional Application No. 60/240,345, filed Oct. 11, 2000, the entire content of which is hereby incorporated by reference.

### INTRODUCTION

Technical Field

The invention relates to new chemical compounds, particularly aryl-indane compounds. The compounds may be used as inhibitors of P-glycoprotein-mediated transport. The compounds are useful for modulation of multidrug resistance during treatment with chemotherapeutic agents and also for enhancing bioavailability of drugs.

### BACKGROUND

Bioavailability

Following oral administration of a drug, the sequence of events that occur include absorption through the various mucosal surfaces, distribution via the blood stream to various tissues, biotransformation in the liver and other tissues, action at the target site, and elimination of drug or metabolites in urine or bile.

Bioavailability of a drug (pharmaceutical composition) following oral dosing can be approximated by the following formula:

Fₒᵣₐₗ=F_{ABS}×F_{G}×FH

Fₒᵣₐₗ is oral bioavailability fraction, which is the fraction of the oral dose that reaches the circulation in an active, unchanged form. Fₒᵣₐₗ is less than 100% of the active ingredient in the oral dose for three reasons: drug is not absorbed through the GI tract and is eliminated in the feces; drug is biotransformed by the cells of the intestine (to an inactive metabolite); or drug is eliminated by the cells of the liver, either by biotransformation and/or by transport into the bile. Thus, oral bioavailability is the product of the fraction of the oral dose that is absorbed (F_{ABS}), the fraction of the absorbed dose that successfully reaches the blood side of the gastrointestinal tract (F_{G}), and the fraction of the drug in the GI blood supply that reaches the heart side of the liver (FH). Previous drug formulations have attempted to increase drug efficacy by increasing drug absorption. For example, methods have been used to increase drug absorption using liposomes as carriers and designing more lipophilic drugs. These methods can increase drug absorption; however, they fail to address other ways of increasing drug bioavailability.

Oral Bioavailability—Absorption By The Gut

Absorption across epithelia, in particular intestinal epithelia, affects drug bioavailability. The intestinal lumen presents a convoluted surface that increases the surface area of the intestine to facilitate absorption of both nutrients and drugs. The membrane of the enterocyte contains many transport proteins that actively carry nutrients from the lumen of the gut into the interior of the enterocytes. Many molecules, including many drugs, passively diffuse or are actively transported through the membrane and into the cytoplasm. Most nutrients and drugs pass through the enterocyte and eventually diffuse into the capillary net en route to the portal circulation system and the liver.

The intestine can also pump drugs out of the intestine and back into the lumen. The ability of the intestine to pump drugs out of the tissue has been thought to be important in protection against potentially damaging hydrophobic cations and toxins and for protection against small intestine cancer. Compounds or formulations to reduce pumping of drugs back into the intestine to increase drug bioavailability are needed.

Multiple Drug Resistance

The phenomenon of multiple drug resistance (MDR) is characterized by cross-resistance of tumor cells to multiple cytotoxic anti-cancer agents that are structurally and mechanistically distinct (Sharom, F. J., “The P-Glycoprotein Efflux Pump: How Does It Transport Drugs,” J. Membrane Biol., 160:161-175 (1997); Germann, U. A., “P-glycoprotein-A Mediator Of Multidrug Resistance In Tumour Cells,” Eur. J. Cancer, 32A:927-944 (1996); Gottesman, M. M., Pastan, I., “Biochemistry Of Multidrug Resistance Mediated By The Multidrug Transporter,” Ann. Rev. Biochem., 62:385-427 (1993); Chin, K-V., Pastan, I., Gottesman, M. M., “Function And Regulation Of The Human Multidrug Resistance Gene,” Adv. Can. Res. 60:157-180 (1993)). Tumors from human cancer patients may have MDR to anticancer drugs prior to exposure to initial therapy or may develop resistance subsequent to treatment. Additionally, MDR may arise to drugs not previously used in the therapeutic regimen.

A major form of MDR arises from the expression of an integral membrane protein, P-glycoprotein (P-gp), which functions as a drug efflux pump. The MDRI gene in humans and the mdr1a and mdr1b genes in rodents and other species encode P-gp. This protein is a trans-membrane protein that functions as an ATP-dependent drug efflux pump removing cytotoxic substrate drugs from the cell preventing their accumulation to toxic levels.

Expressed on the luminal surface of the epithelial cells of excretory organs such as liver, small intestine, and kidney and in the endothelial cells comprising the blood-brain barrier, P-gp is well situated to operate as a barrier against many drugs. P-gp is expressed on the apical surface of intestinal villus enterocytes, where it can determine the absorption of substrate drugs. A role for P-gp in detoxification pathways and limiting uptake of drugs and xenobiotics has been substantiated by experimental observations using both in vitro and in vivo model systems (Silverman, J. A., P-glycoprotein In Metabolic Drug Interactions, eds, R. Levy, K. E. Thummel, W. F. Trager, P. D. Hansten, M. Eichelbaum, Philadelphia, Lippencott Williams & Wilkins, pp.135-144 (2000)).

Since the discovery of the drug efflux activity of P-gp, numerous investigators have attempted to inhibit P-gp-mediated drug efflux with the ultimate goal of increasing the efficacy of cancer chemotherapy. Initial attempts utilized existing compounds however, and due to undesirable pharmacological activities, these studies have had limited success. Additional, more potent, MDR/P-gp reversal agents are needed.

### SUMMARY OF THE INVENTION

The present invention relates to compounds of Formula I:

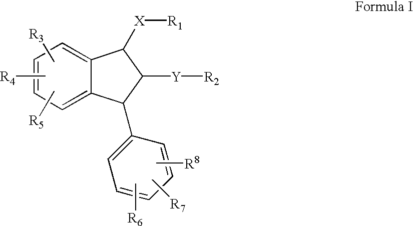

wherein:

R₁ and R₂ are each independently —OR₉ or —NR₁₀R₁₁;

R₃, R₄, R₅, R₆, R₇, and R₈ are each independently hydrogen, C₁₋₁₀ alkyl, C₂₋₁₀ alkenyl, C₂₋₁₀ alkynyl, C₁₋₁₀ alkoxy, phenyl, phenoxy, benzyl, benzyloxy, C₃₋₈ cycloalkyl, N(R₁₂)₂, NHCOR₁₃, S(O)_{q}C₁₋₁₀ alkyl, OH, or halogen; wherein C₁₋₁₀ alkyl, C₂₋₁₀ alkenyl, or C₂₋₁₀ alkynyl may be optionally substituted by COOH, OH, CO(CH₂)ₙCH₃, CO(CH₂)ₙ, CH₂N(R₁₂)₂, or halogen; or R₃ and R₄, R₄ and R₅, R₆ and R₇, or R₇ and R₈ together may be —O—A—O— on contiguous carbons;

R₉ is C₁₋₁₀ alkylene, C₂₋₁₀ alkenylene, C₂₋₁₀ alkylidene, or C₂₋₁₀ alkynylene, all of which may be linear or branched or phenylene, all of which may be unsubstituted or substituted by one or more OH, COOH, alkoxy, NHR₁₂, N(R₁₂)₂, NHCOR₁₃ or halogen or R₉ is alkylsilyl, arylsilyl or alkylarylsilyl;

R₁₀ and R₁₁ are each independently C₁₋₁₀ alkylene, C₁₋₁₀ alkenylene, C₂₋₁₀ alkylidene, C₂₋₁₀ alkynylene, S(O)_{q}(R₁₄), C(O)NH(R₁₄), and C(O)_{q}(R₁₄), all of which may be linear or branched, phenylene or benzylene, all of which may be unsubstituted or substituted by one or more OH, COOH, alkoxy, NHR₁₂, N(R₁₂)₂, NHCOR₁₃ or selected from the following group:

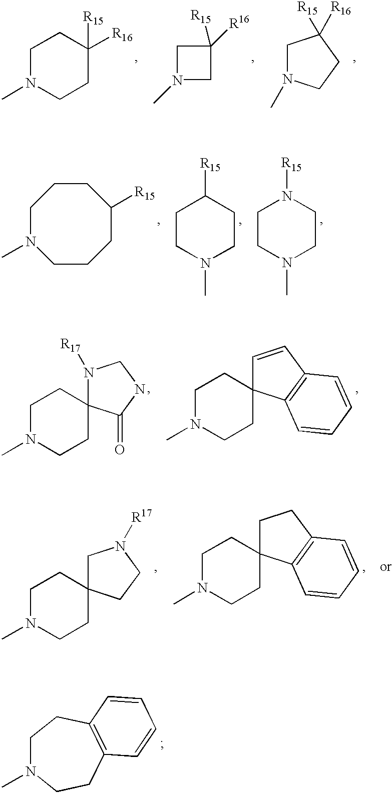

R₁₂ is hydrogen, C₁₋₁₀ alkyl, C₂₋₁₀ alkenyl, benzyl, or aryl, all of which may be unsubstituted or substituted by one or more OH, COOH, NH₂, secondary amine, tertiary amine, tetrazole, or PO₃H₂;

R₁₃ is C₁₋₁₀ alkyl, C₂₋₁₀ alkenyl, benzyl, or aryl, all of which may be unsubstituted or substituted by one or more OH, COOH, NH₂, secondary amine, tertiary amine, tetrazole, or PO₃H₂;

R₁₄ is C₁₋₁₀ alkyl, C₂₋₁₀ alkenyl, C₂₋₁₀ alkynyl, C₃₋₆ cycloalkyl, phenyl, and benzyl; wherein C₁₋₁₀ alkyl, C₂₋₁₀ alkenyl, C₂₋₁₀ alkynyl maybe optionally substituted by COOH, CO(CH₂)ₙCH₃ or OH;

R₁₅ and R₁₆ are each independently hydrogen, aryl, C₁₋₁₀ alkyl, C₂₋₁₀ alkenyl, and C₂₋₁₀ alkynyl, all of which may be unsubstituted or substituted by one or more CH₂OH, N(R₁₂)₂, NHCOR₁₃, OH, or halogen; wherein aryl is naphthyl, indolyl, pyridyl, thienyl, oxazolidinyl, oxazolyl, thiazolyl, isothiazolyl, pyrazolyl, triazolyl, tetrazolyl, imidazolyl, imidazolidinyl, thiazolidinyl, isoxazolyl, oxadiazolyl, thiadiazolyl, morpholinyl, piperidinyl, piperazinyl, pyrrolyl or pyrimidyl, all of which may be unsubstituted or substituted by one or more R₁₇, R₁₈, R₁g; wherein if R₁₅ occurs without R₁₆, R₁₅ is not hydrogen;

R₁₇, R₁₈, R₁₉ are each independently hydrogen, C₁₋₁₀ alkyl, C₂₋₁₀ alkenyl, C₂₋₁₀ alkynyl, phenyl, benzyl, C₃₋₈ cycloalkyl, C₁₋₁₀alkoxy, S(O)_{q}C₁₋₁₀ alkyl, N(R₁₄)₂, NHCOR₆, OH, or halogen; wherein C₁₋₁₀ alkyl, C₂₋₁₀ alkenyl or C₂₋₁₀ alkynyl may be optionally substituted by COOH, CO(CH₂)ₙCH₃, CO(CH₂)ₙ, CH₂N(R₁₄)₂, OH or halogen;

X and Y are each independently —CH₂—, —(CH₂)₂—, —(CH₂)₃—, C═O, CH₂C(O), (CH₂)₂C(O), CH₂SO₂, and (CH₂)₂SO₂;

q is zero, one, two, or three; and

n is an integer from zero to six;

or a diastereomer, enantiomer or pharmaceutically acceptable salts thereof.

One aspect of the invention is the use of the compound of Formula I as an inhibitor of P-glycoprotein-mediated transport. The inhibition of P-gp-mediated transport through use of the compound may take place in intestinal epithelia, tumor cells, or some other context. Another aspect of the invention is a method of using the compound of Formula I to inhibit P-gp-mediated transport.

A method of increasing bioavailability of an orally administered pharmaceutical compound or drug is also an embodiment of the invention. The method comprises coadministering to a mammal a compound of Formula I and a drug or drugs. Drug bioavailability is maximized by inhibiting P-gp-mediated transport of drugs. The Formula I compounds inhibit P-gp-controlled back transport to increase the net transport of drugs through the enterocyte layer, causing an increase in the bioavailability of the drug, since the protein P-gp pumps drugs that have been transported into the cytoplasm of the enterocytes back into the lumen of the gut. For the purpose of this invention, the compounds of Formula I serve as “bioenhancers.”

Another embodiment of the invention relates to a method of modulating multi-drug resistance during treatment with therapeutic agents. The method comprises administering to a mammal a composition including the compound of Formula I and a therapeutic agent or therapeutic agents or, alternatively, coadministering the compound of Formula I and a therapeutic agent or therapeutic agents. This method is particularly advantageous for modulating resistance to chemotherapeutic agents used in the treatment of neoplastic disease.

Another aspect of the invention relates to a method of converting a non-orally bioavailable drug into an orally bioavailable drug by combining the compound of Formula I and the non-orally bioavailable drug.

The compounds of Formula I may also be used in a method of delivering a drug to the central nervous system of a patient.

Yet another aspect of the invention relates to a pharmaceutical composition containing a compound of Formula I in a pharmaceutically acceptable carrier. A further aspect of the invention relates to a pharmaceutical composition containing a compound of Formula I and a therapeutic agent in a pharmaceutically acceptable carrier.

### BRIEF DESCRIPTION OF THE DRAWINGS

FIG. 1 depicts a method for making compounds of Formula I wherein Y is —(CH₂)₃—.

FIG. 2 depicts a method for making compounds of Formula I wherein X is —(CH₂)₂—.

FIG. 3 depicts a method for making compounds of Formula I wherein the substituents of X or Y are sulfates or sulfonamides.

FIG. 4 depicts a method for making compounds of Formula I wherein X or Y —(CH₂)₁ₒᵣ₂— and the substituents of X or Y are ureas or sulfonamides or amides.

FIG. 5 depicts a dose-response study of multidrug resistant cells sensitized to a doxorubicin in the presence of AV-202.

FIG. 6 depicts a dose-response study of inhibition of P-glycoprotein in the presence of AV-202 and vinblastine.

### DETAILED DESCRIPTION OF THE INVENTION

The invention relates to a new family of compounds, i.e., aryl-indane compounds. These compounds act as inhibitors of P-gp-mediated transport. Accordingly, this family of compounds is useful to enhance the oral bioavailability of drugs in mammals, including man. Also, these compounds are useful to modulate multidrug resistance during treatment with therapeutic agents, such as those used for cancer chemotherapy. The novel compounds may also be used to deliver a drug to the central nervous system of a patient. Another benefit is that they can be used to convert a non-orally bioavailable drug into an orally bioavailable drug.

The present invention relates to compounds of Formula I:

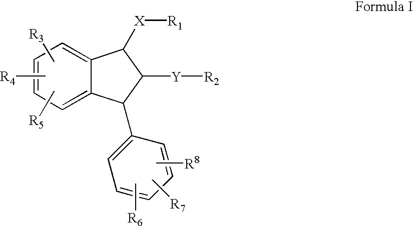

wherein:

R₁ and R₂ are each independently —OR₉ or —NR₁₀R₁₁;

R₃, R₄, R₅, R₆, R₇, and R₈ are each independently hydrogen, C₁₋₁₀ alkyl, C₂₋₁₀ alkenyl, C₂₋₁₀ alkynyl, C₁₋₁₀ alkoxy, phenyl, phenoxy, benzyl, benzyloxy, C₃₋₈ cycloalkyl, N(R₁₂)₂, NHCOR₁₃, S(O)_{q}C₁₋₁₀ alkyl, OH, or halogen; wherein C₁₋₁₀ alkyl, C₂₋₁₀ alkenyl, or C₂₋₁₀ alkynyl may be optionally substituted by COOH, OH, CO(CH₂)ₙCH₃, CO(CH₂)ₙ, CH₂N(R₁₂)₂, or halogen; or R₃ and R₄, R₄ and R₅, R₆ and R₇, or R₇ and R₈ together may be —O—A—O— on contiguous carbons;

R₉ is C₁₋₁₀ alkylene, C₂₋₁₀ alkenylene, C₂₋₁₀ alkylidene, or C₂₋₁₀ alkynylene, all of which may be linear or branched or phenylene, all of which may be unsubstituted or substituted by one or more OH, COOH, alkoxy, NHR₁₂, N(R₁₂)₂, NHCOR₁₃ or halogen; or R₉ is alkylsilyl, arylsilyl or alkylarylsilyl;

R₁₀ and R₁₁ are each independently C₁₋₁₀ alkylene, C₁₋₁₀ alkenylene, C₂₋₁₀ alkylidene, C₂₋₁₀ alkynylene, S(O)_{q}(R₁₄), C(O)NH(R₁₄), and C(O)_{q}(R₁₄), all of which may be linear or branched, phenylene or benzylene, all of which may be unsubstituted or substituted by one or more OH, COOH, alkoxy, NHR₁₂, N(R₁₂)₂, NHCOR₁₃ or selected from the following group:

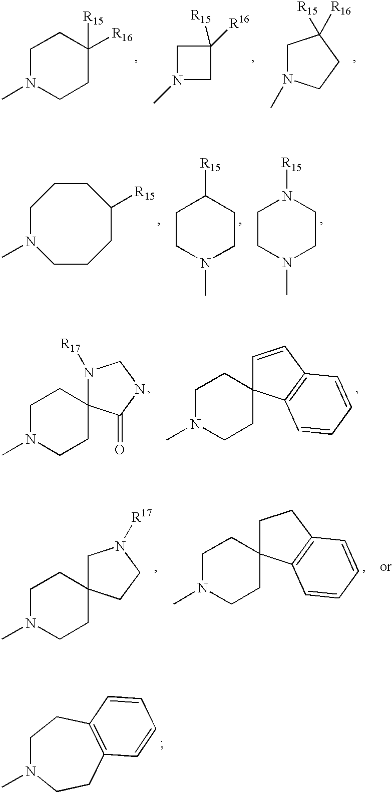

R₁₂ is hydrogen, C₁₋₁₀ alkyl, C₂₋₁₀ alkenyl, benzyl, or aryl, all of which may be unsubstituted or substituted by one or more OH, COOH, NH₂, secondary amine, tertiary amine, tetrazole, or PO₃H₂;

R₁₃ is C₁₋₁₀ alkyl, C₂₋₁₀ alkenyl, benzyl, or aryl, all of which may be unsubstituted or substituted by one or more OH, COOH, NH₂, secondary amine, tertiary amine, tetrazole, or PO₃H₂;

R₁₄ is C₁₋₁₀ alkyl, C₂₋₁₀ alkenyl, C₂₋₁₀ alkynyl, C₃₋₆ cycloalkyl, phenyl, and benzyl; wherein C₁₋₁₀ alkyl, C₂₋₁₀ alkenyl, C₂₋₁₀ alkynyl maybe optionally substituted by COOH, CO(CH₂)ₙCH₃ or OH;

R₁₅ and R₁₆ are each independently hydrogen, aryl, C₁₋₁₀ alkyl, C₂₋₁₀ alkenyl, and C₂₋₁₀ alkynyl, all of which may be unsubstituted or substituted by one or more CH₂OH, N(R₁₂)₂, NHCOR₁₃, OH, or halogen; wherein aryl is naphthyl, indolyl, pyridyl, thienyl, oxazolidinyl, oxazolyl, thiazolyl, isothiazolyl, pyrazolyl, triazolyl, tetrazolyl, imidazolyl, imidazolidinyl, thiazolidinyl, isoxazolyl, oxadiazolyl, thiadiazolyl, morpholinyl, piperidinyl, piperazinyl, pyrrolyl or pyrimidyl, all of which may be unsubstituted or substituted by one or more R₁₇, R₁₈, R₁₉; wherein if R₁₅ occurs without R₁₆, R₁₅ is not hydrogen;

R₁₇, R₁₈, R₁₉ are each independently hydrogen, C₁₋₁₀ alkyl, C₂₋₁₀ alkenyl, C₂₋₁₀ alkynyl, phenyl, benzyl, C₃₋₈ cycloalkyl, C₁₋₁₀ alkoxy, S(O)_{q}C₁₋₁₀ alkyl, N(R₁₄)₂, NHCOR₆, OH, or halogen; wherein C₁₋₁₀ alkyl, C₂₋₁₀ alkenyl or C₂₋₁₀ alkynyl may be optionally substituted by COOH, CO(CH₂)ₙCH₃, CO(CH₂)ₙ, CH₂N(R₁₄)₂, OH or halogen;

X and Y are each independently —CH₂—, —(CH₂)₂—, —(CH₂)₃—, C═O, CH₂C(O), (CH₂)₂C(O), CH₂SO₂, and (CH₂)₂SO₂;

q is zero, one, two, or three; and

n is an integer from zero to six.

Also included in the invention are pharmaceutically acceptable salts of the compounds of Formula I. Pharmaceutically acceptable salts include both the metallic (inorganic) salts and the organic salts, a list of which is given in Remington's Pharmaceutical Sciences, A. R. Gennaro, et al., eds., 18th edition, p. 1444-45 (1990). It is well known to one skilled in the art that an appropriate salt form is chosen based on physical and chemical stability, flowability, hydroscopicity, and solubility. Examples of pharmaceutically acceptable salts include salts with inorganic acids such as hydrochloric acid, hydrobromic acid, phosphoric acid, diphosphoric acid, nitric acid, and sulfuric acid. Examples of salts with organic acids include as methanesulfonic acid, p-toluenesulfonic acid, acetic acid, fumaric acid, succinic acid, lactic acid, mandelic acid, malic acid, or maleic acid, citric acid, tartaric acid, palmitic acid, salicylic acid and stearic acid. In addition, if the compound of Formula I contains a carboxy group, it may be converted into a pharmaceutically acceptable addition salt with inorganic or organic bases. Examples of suitable bases include sodium hydroxide, potassium hydroxide, calcium hydroxide, lithium hydroxide, ammonia, cyclohexylamine, dicyclohexylamine, ethanolamine, diethanolamine and triethanolamine. Preferred salts of this invention are potassium, sodium, calcium and ammonium salts. Also included within the scope of this invention are any of various crystal forms, hydrates and solvates of the compound of Formula I.

The term alkylene is a divalent alkyl group in which the bonds are on two different carbon atoms; alkylidene is a divalent alkyl group in which the bonds are on the same carbon atom; alkenylene is a divalent alkene group in which the bonds may be on any carbon atom; alkynylene is a divalent alkynyl group in which the bonds may be on any carbon atom. All alkyl, alkenyl, alkynyl, alkoxy, alkylene, alkylidene, alkenylene, and alkynylene groups may be straight or branched. The term halogen is used to mean iodo, fluoro, chloro, or bromo. Alkyl groups may be substituted by one or more halogens up to perhalogenation. A secondary amine is a nitrogen radical bonded to one hydrogen and to one carbon atom. A tertiary amine is a nitrogen radical bonded to carbon atoms.

The compounds of the present invention may contain one or more asymmetric carbon atoms and may exist in racemic and optically active form. Compounds with carbon-carbon double bonds may occur in Z and E form with all isomeric forms of the compounds being included in the present invention. All of these isomers, enantiomers, tautomers, and diastereomers are contemplated to be within the scope of present invention.

Preferred compounds are those according to Formula I wherein X and Y are —CH₂—, —(CH₂)₂—, —(CH₂)₃—, C═O, or CH₂C(O); R₃, R₄, R₅, R₆, R₇, and R₈ are C₁₋₁₀ alkoxy or benzyloxy; and R₉, R₁₀, or R₁₁, are the following as appropriate: 4-benzylpiperidinyl, 1-benzylpiperazinyl, 1-phenylpiperazinyl, 1-(4-piperazin-1-yl-phenyl)-ethanone, 1-(4-chlorophenyl)-piperazinyl, 1-(4-methoxyphenyl)-piperazinyl, 1-(4-fluorophenyl)-piperazinyl, 2-piperazin-1-yl-pyrimidinyl, 1-(2-ethoxyphenyl)-piperazinyl, 1-(3-phenylallyl)-piperazinyl, benzylethylamino, piperazinyl-1-carboxylic acid ethyl ester, piperazinyl-1-carboxylic acid t-butyl ester, piperazinyl-1-carboxylic acid benzyl ester, dimethylamino, diethylamino,1-ethylpiperazinyl, 1-(2-methoxyphenyl)-piperazinyl, dipropylamino, dibutylamino, diisobutylamino, bis-(2-ethylhexyl)-amino, dihexylamino,1-pyridin-2-yl-piperazinyl, 1-(4-nitrophenyl)-piperazinyl, benzenesulfonyl, naphthalene-2-sulfonyl, 4-chlorobenzenesulfonyl, 4-methoxybenzenesulfonyl, butane-1-sulfonyl, 4-nitrobenzenesulfonyl, octanesulfonyl, 3,4-dimethoxybenzenesulfonyl, and isocyanate-benzenyl.

Preferred compounds are the following:

(AV-159), 2-[2-(4-benzylpiperidine-1-carbonyl)-3-(3,4-dimethoxyphenyl)-5,6-dimethoxy-indan-1-yl]-1-(4-benzylpiperidin-1-yl)-ethanone;

(AV-160), 2-[2-(4-benzylpiperidin-1-yl)-methyl-3-(3,4-dimethoxyphenyl)-5,6-dimethoxy-indan-1-yl]-1-(4-benzylpiperidin-1-yl)-ethane;

(AV-161), 2-[3-(3,4-dimethoxyphenyl)-5,6-dimethoxy-2-(4-phenylpiperazin-1-carbonyl)-indan-1-yl]-1-(4-phenylpiperazin-1-yl)-ethanone;

(AV-162), 2-[3-(3,4-dimethoxyphenyl)-5,6-dimethoxy-2-(4-phenylpiperazin-1-yl) methyl-indan-1-yl]-1-(4-phenylpiperazin-1-yl)-ethane;

(AV-163), 2-[2-[4-(4-acetylphenyl)-piperazin-1-carbonyl]-3-(3,4-dimethoxyphenyl)-5,6-dimethoxy-indan-1-yl]-1[4-(4-acetylphenyl)piperazine-1-yl]-ethanone;

(AV-164), 2-[2-(4-benzylpiperazine-1-carbonyl)-3-(3,4-dimethoxyphenyl)-5,6-dimethoxy-indan-1-yl]-1-(4-benzylpiperazine-1-yl)-ethanone;

(AV-167), 2-[2-[4-(4-chlorophenyl)-piperazin-1 carbonyl]-3-(3,4-dimethoxyphenyl)-5,6-dimethoxy-indan-1-yl]-1-[4-(4-chlorophenyl)piperazine-1-yl]-ethanone;

(AV-168), 2-{3-(3,4-dimethoxyphenyl)-5,6-dimethoxy-2-[4-(4-methoxyphenyl)-piperazin-1-carbonyl]-indan-1-yl}-1-[4-(4-methoxyphenyl)-1-yl]-ethanone;

(AV-169), 2-{2-[4-(4-fluorophenyl)-piperazin-1-carbonyl]-3-(3,4-dimethoxyphenyl)-5,6-dimethoxy-indan-1-yl}-1-[4-(4-fluorophenyl)piperazine-1-yl]-ethanone;

(AV-170), 2-{3-(3,4-dimethoxyphenyl)-5,6-dimethoxy-2-(pyrimidin-2-yl-piperazin-1-carbonyl)-indan-1-yl]-1-(pyrimidin-2-yl-piperazine-1-yl)-ethanone;

(AV-171), 2-{3-(3,4-dimethoxyphenyl)-5,6-dimethoxy-2-[4-(2-ethoxyphenyl)-piperazin-1-carbonyl]-indan-1-yl}-1-[4-(2-ethoxyphenyl)-1-yl]-ethanone;

(AV-172), 2-{3-(3,4-dimethoxyphenyl)-5,6-dimethoxy-2-[4-(3-phenylallyl)-piperazine-1-carbonyl]-indan-1-yl}-1-[4-(3-phenylallyl)piperazine-1-yl]-ethanone;

(AV-173), 1-[(benzylethylcarbamoyl)-methyl]-3-(3,4-dimethoxyphenyl)-5,6-dimethoxy-indan-2-carboxylic acid benzyl ethyl amide; (AV-174), 4-{2-[3-(3,4-dimethoxyphenyl)-5,6-dimethoxy-2-(piperazine-1-carboxylic acid ethyl ester)-indan-1-yl]-acetyl}-piperazine-1-carboxylic acid ethyl ester;

(AV-175), [1-[2-t-butyldiphenyl-silanoxy)-ethyl-3-(3,4-dimethoxyphenyl)-5,6-dimethoxy-indan-2-yl]-methanol;

(AV-176), 2-[3-[3,4-dimethoxyphenyl)-5,6-dimethoxy-2-methoxymethyl-indan-1-yl]-ethanol;

(AV-177), methanesulfonic acid 2-[3-(3,4-dimethoxyphenyl)-5,6-dimethoxy-2-methoxymethyl-indan-1-yl] ethyl ester;

(AV-178), 1-benzyl-4-{2-[3-(3,4-dimethoxyphenyl)-5,6-dimethoxy-2-methoxymethyl-indan-1-yl]-ethyl}-piperazine;

(AV-179), 4-{2-[3-(3,4-dimethoxyphenyl)-5,6-dimethoxy-2-(piperazine-1-carboxylic acid benzyl ester)-indan-1-yl]-acetyl}-piperazine-1-carboxylic acid benzyl ester;

(AV-180), 4-{2-[3-(3,4-dimethoxyphenyl)-5,6-dimethoxy-2-(piperazine-1-carboxylic acid t-butyl ester)-indan-1-yl]-acetyl}-piperazine-1-carboxylic acid t-butyl ester;

(AV-181),1-(3,4-dimethoxyphenyl)-3-dimethylcarbamoylmethyl-5,6-dimethoxy-indan-2-carboxylic acid dimethyl amide;

(AV-182), 1-(3,4-dimethoxyphenyl)-3-diethylcarbamoylmethyl-5,6-dimethoxy-indan-2-carboxylic acid diethyl amide;

(AV-183), 2-{3-(3,4-dimethoxyphenyl)-5,6-dimethoxy-2-[4-(4-methoxy)-piperazin-1-yl-methyl]-indan-1-yl}-1-[4-(4-methoxyphenyl)-piperazin-1-yl]-ethane;

(AV-184), 2-{3-(3,4-dimethoxyphenyl)-5,6-dimethoxy-2-[4-(2-ethoxyphenyl)-piperazin-1-yl-methyl]-indan-1-yl}-1-[4-(2-ethoxyphenyl)-piperazin-1-yl]-ethane;

(AV-185), 2-[3-(3,4-dimethoxyphenyl)-5,6-dimethoxy-2-(piperazine-1-carbonyl)-indan-1-yl]-1-piperazin-1-yl-ethanone;

(AV-186), 2-{3-(3,4-dimethoxyphenyl)-5,6-dimethoxy-2-[4-(4-chlorophenyl)-piperazin-1-yl-methyl]-indan-1-yl}-1-[4-(4-chlorophenyl)-piperazin-1-yl]-ethane;

(AV-187), 2-[3-(3,4-dimethoxyphenyl)-5,6-dimethoxy-2-(4-ethylpiperazine-1-carbonyl)-indan-1-yl]-1-(4-ethylpiperazin-1-yl)-ethanone;

(AV-188), 2-{3-(3,4-dimethoxyphenyl)-5,6-dimethoxy-2-[4-(2-methoxyphenyl)-piperazin-1-carbonyl]-indan-1-yl}-1-[4-(2-methoxy-phenyl)-1-yl]-ethanone;

(AV-189), 1-(3,4-dimethoxyphenyl)-3-dipropylcarbamoylmethyl-5,6-dimethoxy-indan-2-carboxylic acid dipropyl amide;

(AV-190),1-(3,4-dimethoxyphenyl)-3-dibutylcarbamoylmethyl-5,6-dimethoxy-indan-2-carboxylic acid dibutyl amide;

(AV-191),1-[(diisobutylcarbamoyl)methyl]-3-(3,4-dimethoxyphenyl)-5,6-dimethoxy-indan-2-carboxylic acid diisobutylamide;

(AV-192), 1-{[bis(2-ethylhexyl)carbamoyl]methyl}-3-(3,4-dimethoxyphenyl)-5,6-dimethoxy-indan-2-carboxylic acid bis(2-ethylhexyl) amide;

(AV-193), 1-(3,4-dimethoxyphenyl)-3-dihexylcarbamoylmethyl-5,6-dimethoxy-indan-2-carboxylic acid dihexyl amide;

(AV-194),1 {-2-[3-(3,4-dimethoxyphenyl)-5,6-dimethoxy-2-methoxymethyl-indan-1-yl]-ethyl}-4-(2-ethoxyphenyl)-piperazine;

(AV-195), 2-{3-(3,4-dimethoxyphenyl)-5,6-dimethoxy-2-(pyridin-2-yl-piperazin-1-carbonyl)-indan-1-yl]-1-(pyridin-2-yl-piperazine-1-yl)-ethanone;

(AV-196), 2-{3-(3,4-dimethoxyphenyl)-5,6-dimethoxy-2-[4-(4-nitrophenyl)-piperazin-1-carbonyl]-indan-1-yl}-1-[4-(4-nitrophenyl)-1-yl]-ethanone;

(AV-197), 1-benzyl-4-{2-[3-(3,4-d imethoxyphenyl)-5,6-dimethoxy-2-methoxymethyl-indan-1-yl]-ethyl}-piperidine;

(AV-198), 1-{-2-[3-(3,4-dimethoxyphenyl)-5,6-dimethoxy-2-methoxymethyl-indan-1-yl]-ethyl}-4-phenylpiperazine;

(AV-199), 1-{-2-[3-(3,4-dimethoxyphenyl)-5,6-dimethoxy-2-methoxymethyl-indan-1-yl]-ethyl}-4-(4-methoxyphenyl)-piperazine;

(AV-200), 4-{2-[3-(3,4-dimethoxyphenyl)-5,6-dimethoxy-2-methoxymethyl-indan-1-yl]-ethyl}-piperazine-1-carboxylic acid benzyl ester;

(AV-201),1-{-2-[3-(3,4-dimethoxyphenyl)-5,6-dimethoxy-2-methoxymethyl-indan-1-yl]-ethyl}-4-(4-fluorophenyl)-piperazine;

(AV-202), 1{-2-[3-(3,4-dimethoxyphenyl)-5,6-dimethoxy-2-methoxymethyl-indan-1-yl]-ethyl}-4-(2-methoxyphenyl)-piperazine; (AV-203), 1-[4-(4-{2-[3-3,4-dimethoxyphenyl)-5,6-dimethoxy-2-methoxymethyl-indan-1-yl]-ethyl}-piperazin-1-yl)-phenyl]-ethanone;

(AV-204), 1-{2-[3-(3,4-dimethoxyphenyl)-5,6-dimethoxy-2-methoxymethyl-indan-1-yl]-ethyl}-4-(3-phenylallyl)-piperazine;

(AV-205), 2-{5-benzyloxy-3-(4-benzyloxy-3-methoxyphenyl)-2-{4-(2-ethoxyphenyl)-piperazine-1-carbonyl}-6-methoxy-indan-1-yl}-1-{4-(2-ethoxyphenyl)-piperazin-1-yl]-ethanone;

(AV-206), 2-[2-[4-(2-ethoxyphenyl)-piperazine-1-carbonyl]-5-hydroxy-3-(4-hydroxy-3-methoxyphenyl)-6-methoxy-indan-1-yl]-1-[4(2-ethoxyphenyl)-piperazin-1-yl]-ethanone;

(AV-207), 2-{5-ethoxy-3-(4-ethoxy-3-methoxyphenyl)-2-[4-(2-ethoxyphenyl)-piperazine-1-carbonyl]-6-methoxy-indan-1-yl}-1-[4-(2-ethoxyphenyl)-piperazin-1-yl]-ethanone;

(AV-208), 2-{5-butoxy-3-(4-butoxy-3-methoxyphenyl)-2-[4-(2-ethoxyphenyl)-piperazine-1-carbonyl]-6-methoxy-indan-1-yl}-1-[4-(2-ethoxyphenyl)-piperazin-1-yl]-ethanone;

(AV-209), 2-{5-decyloxy-3-(4-decyloxy-3-methoxyphenyl)-2-[4-(2-ethoxyphenyl)-piperazine-1-carbonyl]-6-methoxy-indan-1-yl}-1-[4-(2-ethoxyphenyl)-piperazin-1-yl]-ethanone;

(AV-223), N-{2-[(2-N-methyl)-benzenesulfonamide-3-(3,4-dimethoxyphenyl)-5,6-dimethoxy-indan-1-yl}-ethyl}-benzensulfonamide; and

(AV-234), 10-benzyl-3-{2-[2-(3-benzyl-ureidomethyl)-3-(3,4-dimethoxyphenyl)-5,6-dimethoxy-indan-1-yl]-ethyl}-urea.

(AV-239-1), 2-[2-4-(2-ethoxy-phenyl)-piperazine-1-carbonyl]-6-methoxy-3-(3-methoxy-4-propoxy-phenyl)-5-propoxy-inda-1-yl]-1-[4-2(ethoxy-phenyl)-piperazin-1-yl]-ethanone

(AV-250), 1-(3,4-Dimethoxy-phenyl)-3-{2-[4-(2-ethoxy-phenyl)-piperazin-1-yl]-2-oxo-ethyl}-5,6-dimethoxy-indan-2-carboxylic acid

(AV-251), 2-[2-(4-Benzyl-piperidine-1-carbonyl)-5-hydroxy-3-(4-hydroxy-3-methoxy-phenyl)-6-methoxy-indan-1-yl-1-(4-benzyl-piperidin-1-yl)-ethanone

(AV-252), 2-[2-[4-(2-Ethoxy-phenyl)-piperazine-1-carbonyl]-6-methoxy-3-(3-methoxy-4-pentyloxy-phenyl)-5-pentyloxy-indan-1-yl]-1-[4-(2-ethoxy-phenyl)-piperazin-1-yl]ethanone

(AV-253), 2-{3-93,4-diethoxy-phenyl)-5,6-diethoxy-2-[4-(2-ethoxy-phenyl)-piperazine-1-carbonyl]-indan-1-yl}-1 [4-2-ethoxy-phenyl)-piperazine-1-yl]-ethanone

(AV-254), 2-{3-(3,4-Dipopoxy-phenyl)-2-[4-(2-ethoxy-phenyl)-piperazine-1-carbonyl]-5,6-dipropoxy-indan-1-yl}-1-[4-(2-ethoxy-phenyl)-piperazin-1-yl]-ethanone

(AV-255), 2-{3-(3,4-Dibutoxy-phenyl)-2-[4-(2-ethoxy-phenyl)-piperazine-1-carbonyl]-5,6-dibutoxy-indan-1-yl}-1-[4-(2-ethoxy-phenyl)-piperazin-1-yl]-ethanone

(AV-256), 2-[3-(3,4-Dimethoxy-phenyl)-5,6-dimethoxy-2-(4-methyl-piperazine-1-carbonyl)-indan-1-yl]-1-[4-2-ethoxy-phenyl)-piperazin-1-yl]-ethanone

(AV-257), 1-(3,4-Dimethoxy-phenyl)-3-{2-[4-(2-ethoxy-phenyl)-piperazin-1-yl]-2-oxo-ethyl}-5,6-dimethoxy-indan-2-carboxylic acid dimethylamide

(AV-258), 2-{7-benzo[1,3]dioxol-5-yl-6-[4-(2-ethoxy-phenyl)-piperazin-1-carbonyl]-6,7-dihydro-5H-indeno[5,6-d][1,3]dioxol-5-yl}-1-[4-(2-ethoxy-phenyl)-piperazin-1-yl]-ethanone

(AV-259), N-{2-[(2-N-methyl)-4-tiert-butyl-benzamide-3(3,4-dimethoxy-phenyl)-5,6-dimethoxy-indan-1-yl}-ethyl-4-tert-butyl-benzamide

(AV-260), N-{2-[(2-N-methyl)-4-trifluoromethoxy-benzamide-3(3,4-dimethoxy-phenyl)-5,6-dimethoxy-indan-1-yl}-ethyl-4-trifluoromethoxy-benzamide

(AV-262), N-{2-[(2-N-methyl)-4-propyl-benzamide-3(3,4-dimethoxy-phenyl)-5,6-dimethoxy-indan-1-yl}-ethyl-4-propyl-benzamide

(AV-263), N-{2-[(2-N-methyl)-4-bromo-benzamide-3(3,4-dimethoxy-phenyl)-5,6-dimethoxy-indan-1-yl}-ethyl-4-bromo-benzamide

(AV-264), N-{2-[(2-N-methyl)-benzamide-3(3,4-dimethoxy-phenyl)-5,6-dimethoxy-indan-1-yl}-ethyl-benzamide

(AV-265), N-{2-[(2-N-methyl)-2-methoxy-benzamide-3(3,4-dimethoxy-phenyl)-5,6-dimethoxy-indan-1-yl}-ethyl-2-methoxy-benzamide

Especially preferred compounds are the following:

(AV-159), 2-[2-(4-benzylpiperidine-1-carbonyl)-3-(3,4-dimethoxyphenyl)-5,6-dimethoxy-indan-1-yl]-1-(4-benzylpiperidin-1-yl)-ethanone;

(AV-171), 2-{3-(3,4-dimethoxyphenyl)-5,6-dimethoxy-2-[4-(2-ethoxyphenyl)-piperazin-1-carbonyl]-indan-1-yl}-1-[4-(2-ethoxyphenyl)-1-yl]-ethanone; and

(AV-178), 1-benzyl-4-{2-[3-(3,4-dimethoxyphenyl)-5,6-dimethoxy-2-methoxymethyl-indan-1-yl]-ethyl}-piperazine.

(AV-208), 2-{5-butoxy-3-(4-butoxy-3-methoxyphenyl)-2-[4-(2-ethoxyphenyl)-piperazine-1-carbonyl]-6-methoxy-indan-1-yl}-1-[4-(2-ethoxyphenyl)-piperazin-1-yl]-ethanone.

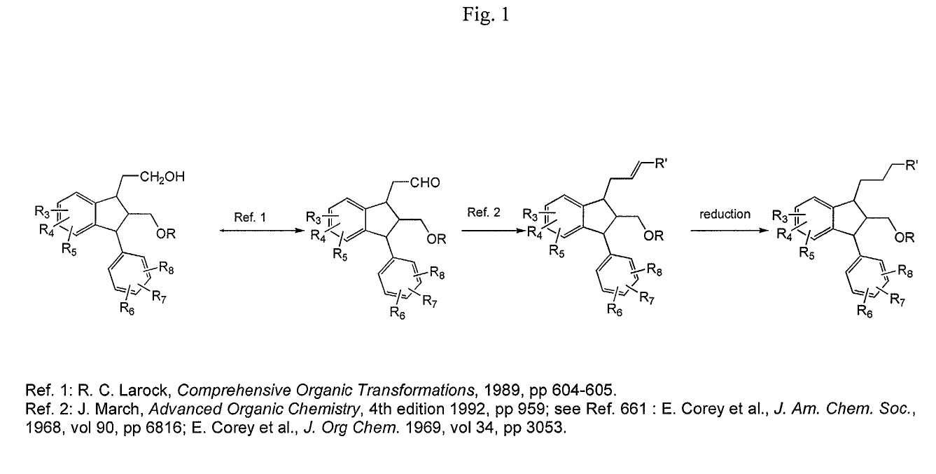

Compounds of Formula I may be prepared, for example, as described below in Scheme 1, Scheme 2, and Scheme 3, and in FIGS. 1-4. According to Scheme 1, a compound of formula (2), wherein w is H, m is 0-3, and p is 1-4, is reacted with a primary or secondary amine of formula (3). The compound of formula (2) may be obtained by methods known to those skilled in the art according to the procedure described by E. Al-Farhan et. al., in Synthesis, (Oct. 1992), pp. 959-961. The reaction with compound (3) is carried out in a suitable solvent such as tetrahydrofuran with a suitable base such as Hunnings base or triethylamine, with a suitable coupling reagent such as 1-ethyl-3-(3-dimethylbutylpropyl)carbodiimide or dicylcohexyl-carbodiimide in presence of a catalyst such as 4-N,N-dimethylamino-pyridine at room temperature to provide a compound of formula (4). Specific examples of a compound of formula (4) that are included in the present invention comprise AV-159, AV-161, AV-163, AV-164, AV-167, AV-168, AV-169, AV-170, AV-171, AV-172, AV-173, AV-174, AV-179, AV-180, AV-181, AV-182, AV-185, AV-187, AV-188, AV-189, AV-190, AV-191, AV-192, AV-193, AV-195, AV-205, AV-206, AV-207, AV-208, AV-209, AV-239, AV-250, AV-251, AV-252, AV-253, AV-254, AV-255, AV-256, AV-257, and AV-258.

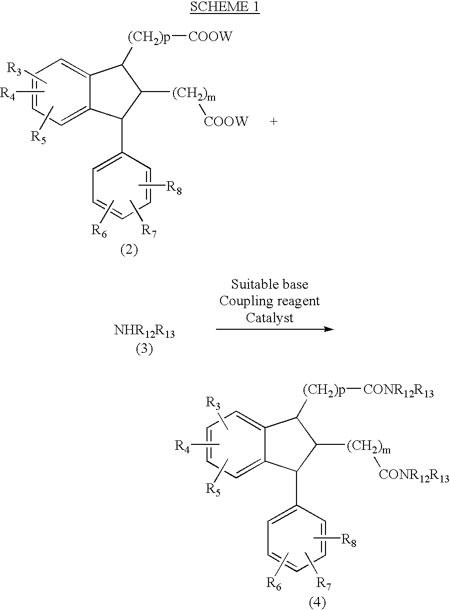

According to Scheme 2, reduction of compounds of formula (4) with a reducing agent, such as lithium aluminum hydride, in a suitable solvent, such as tetrahydrofuran, provides compounds of formula (5). Specific examples of a compound of formula (5) that are included in the present invention comprise AV-160, AV-162, AV-183, AV-184, and AV-186.

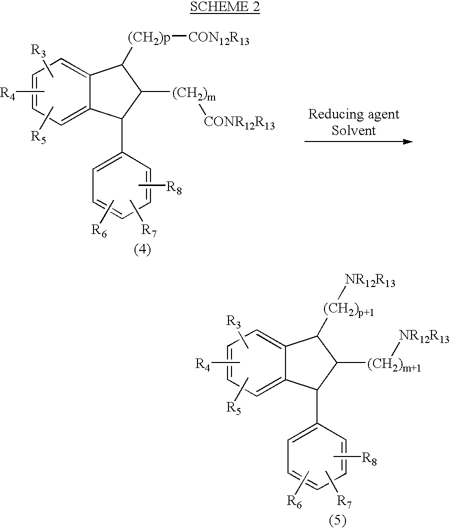

According to Scheme 3, a compound of formula (2), wherein m is 0-3, p is 1-4, and W is C₁₅ alkyl is reduced to provide a compound of formula (6). This reaction is carried out with a suitable reducing agent, such as lithium hydride, in a suitable solvent, such as tetrahydrofuran, at temperatures between -20° C. and 200° C., preferably at 60° C. Next, the hydroxyl groups of formula (6) are mono protected with a suitable protecting group as described in Protective Groups in Organic Synthesis, ₃^{rd} edition, page 141, such as with a t-butyldiphenylsilyl group. This particular protecting group, for example, is introduced by reacting the alcohol with t-butyl diphenylsilyl chloride, in the presence of a suitable base, such as imidazole, in a suitable solvent, such as dichloromethane, at room temperature. This provides a compound of formula (7), wherein e, f, and g are each independently C₁₋₅ alkyl or phenyl, as shown in Scheme 3A.

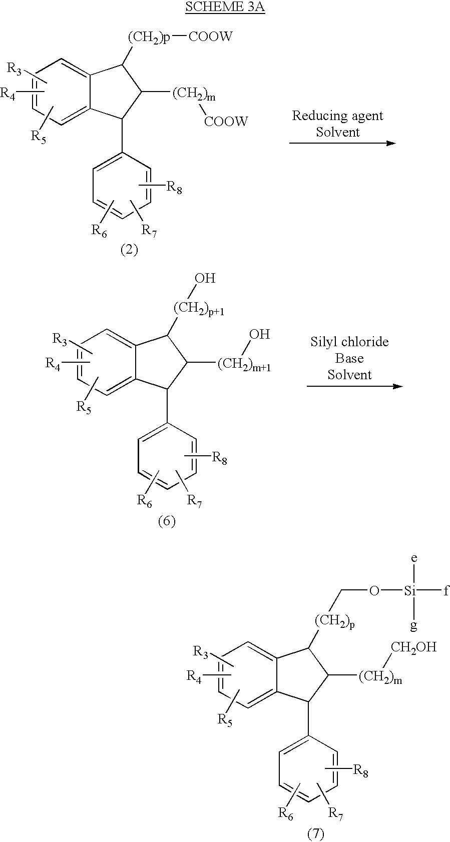

Next, the compound of formula (7) is alkylated. Preferably, the alcohol of formula

(7) is reacted with a suitable alkyl triflate, such as methyl triflate, in presence of suitable base, such as 2,6-di-t-butylpyridine, in a suitable solvent, such as dichloromethane, at room temperature to provide compound of formula (8). Alternatively, a compound of formula (8) may be obtained by heating a mixture of a compound of formula (7), the appropriate alkylhalide, and an excess of silver oxide in an aprotic inert solvent such as dimethylformamide at room temperature. The compound of formula (8) is reacted with a deprotecting agent, such as t-butyl ammonium fluoride, in a solvent, such as tetrahydrofuran, at room temperature to provide a compound of formula (9) as shown in Scheme 3B.

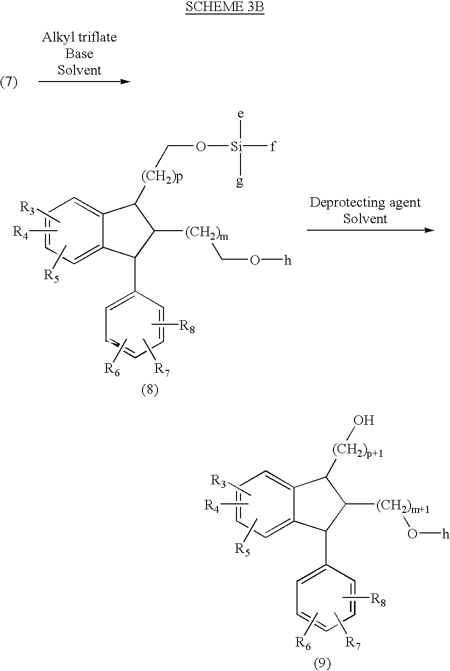

A compound of formula (9) is reacted with methanesulfonic anhydride or p-toluene sulfonyl chloride in the presence of a base, such as pyridine in a suitable solvent, such as dichloromethane, at a temperature between −20° C. and 150° C., but preferably at room temperature, to provide compounds of formula (10). Compounds of formula (10) are reacted with different primary and secondary amines (compounds of formula 3 in Scheme 1) in presence of a base, such as pyridine, diisopropylethylamine, diethylamine, potassium carbonate, or cesium carbonate, in a suitable solvent, such as acetone dimethylformamide, to provide compounds of formula (11), as shown in Scheme 3C. Specific examples of a compound of formula (11) that are included in the present invention comprise AV-178, AV-194, AV-197, AV-198, AV-199, AV-200, AV-201, AV-202, AV-203, and AV-204.

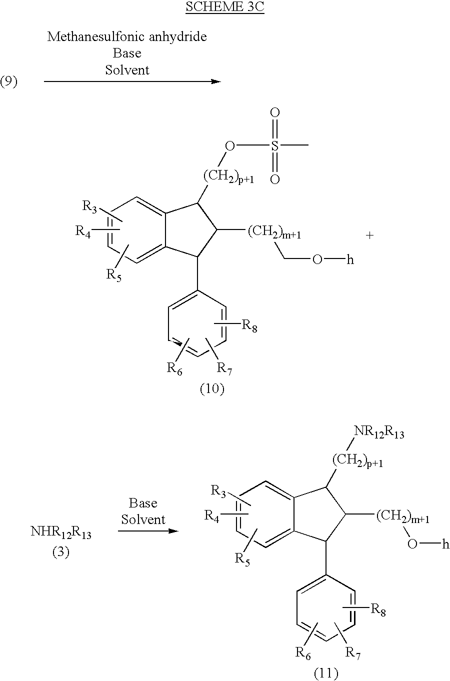

Compounds of Formula I wherein Y is —(CH₂)₃— can be made according to the method shown in FIG. 1 from starting materials described herein.

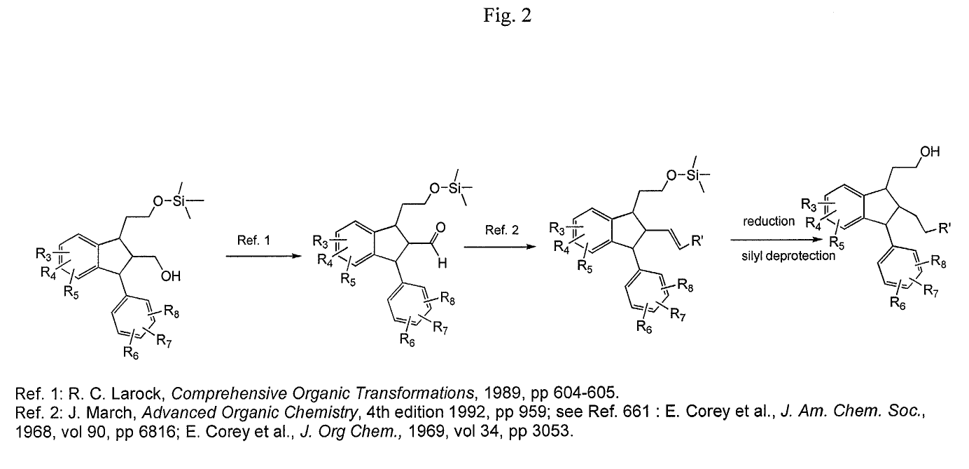

Compounds of Formula I wherein X is —(CH₂)2— can be made according to the method shown in FIG. 2 from starting materials described herein.

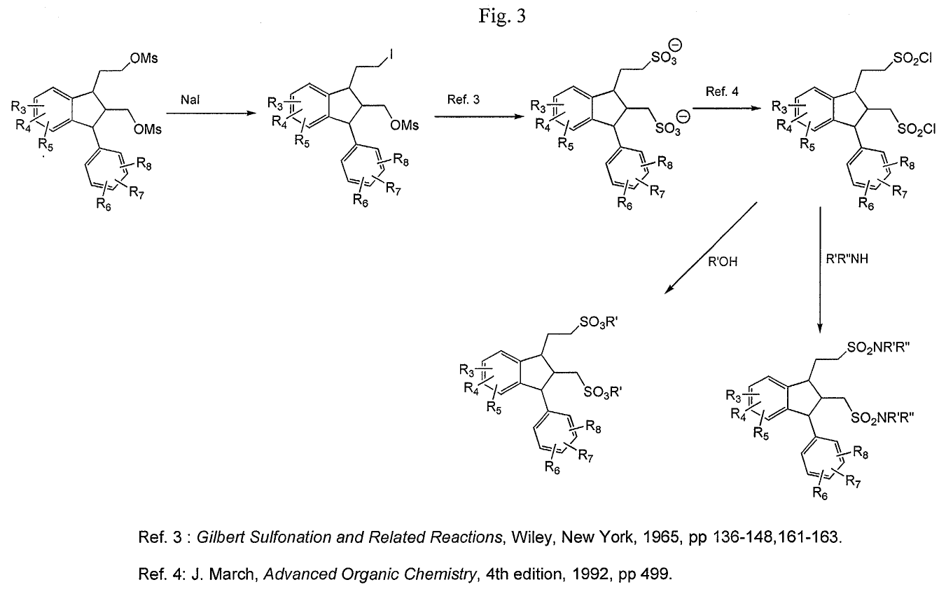

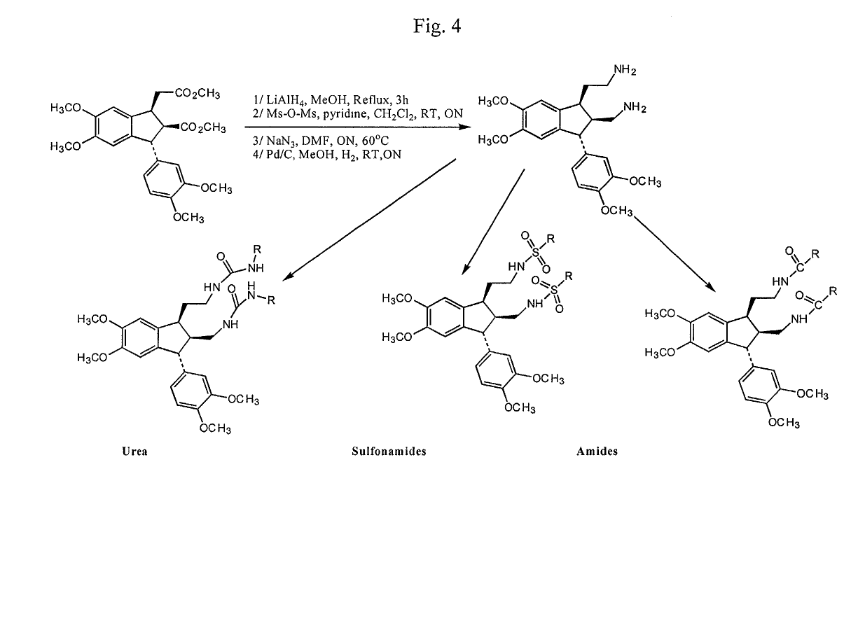

Compounds of Formula I containing sulfonamides and sulfates, specifically AV-223, for example, can be made according to FIGS. 3 and 4.

Compounds of Formula I containing ureas, specifically AV-234, can be made according to FIG. 4.

Compounds of Formula I containing carbonyls, specifically AV-259, AV-260, AV-262, AV-263, AV-264, and AV-265, can be made according to FIG. 4.

Inhibition of P-glycoprotein (P-pq)-Mediated Transport

The invention includes a method of inhibiting P-glycoprotein-mediated transport, specifically by administering a compound of Formula I. Preferably, the compound is selected from the group presented in Table I, shown in Example 46 below. The preferred compounds of the invention have a percentage inhibition of Rhodamine 123 transport of at least 30%, more preferably at least 50%, and most preferably at least 80%.

By using the method of the invention with a coadministered pharmaceutical compound, the coadministered pharmaceutical compound has an increase in cytotoxicity value relative to its cytotoxicity value when administered alone. Preferably this percentage increase in cytotoxicity value is at least 30%, more preferably at least 50%, and most preferably at least 80%.

The invention also includes the use of the compound of Formula I as an inhibitor of P-glycoprotein-mediated transport.

Bioenhancers Increase Drug Bioavailability

An aspect of the present invention is based on new chemical entities affecting drug bioavailability. “Drug bioavailability” is defined as the total amount of drug systemically available over time. The compounds of the invention increase drug bioavailability by inhibiting active transport systems in the gut which decrease the net transport of drugs across gut epithelia. The chemical entity responsible for increased drug bioavailability may be called a bioenhancer. It has been discovered that, in contrast to previous teachings about the primacy of liver metabolism, the gut is also a major location of drug transformation for many drugs and is the primary site of transformation of many orally administered drugs. Thus, bioenhancers specifically targeted to the gut provide a number of advantages, as described in detail below.

In general, an aspect of the present invention provides a method for increasing the bioavailability of an orally administered pharmaceutical compound (particularly one which is hydrophobic), which comprises orally coadministering (1) the pharmaceutical compound or drug to a mammal in need of treatment with (2) a bioenhancer, specifically a compound of Formula I. The bioenhancer is an inhibitor of P-glycoprotein-mediated transport and is present in sufficient amount to provide integrated systemic concentrations over time of the compound greater than the integrated systemic concentrations over time of the compound in the absence of the composition. Changes in the integrated systemic concentrations over time are indicated by the area under the curve (AUC) defined below. In preferred embodiments, side effects are reduced by providing a bioenhancer that is active only (or primarily) in the gut, either because of its structure or absorption characteristics, or because of deliberately selected concentration effects.

Bioavailability Measurements

The increase in drug bioavailability attributable to administration of the bioenhancer can be determined by measuring total systemic drug concentrations over time after coadministration of a drug and a bioenhancer of Formula I and after administration of only the drug. The increase in drug bioavailability is defined as an increase in the Area Under the Curve (AUC). AUC is the integrated measure of systemic drug concentrations over time in units of mass-time/volume. The AUC from time zero (the time of dosing) to time infinity (when no drug remains in the body) following the administration of a drug dose is a measure of the exposure of the patient to the drug. When efficacy of the bioenhancer is being measured, the amount and form of active drug administered should be the same in both the coadministration of drug and bioenhancer and the administration of the drug alone. For instance, administration of 10 mg of drug alone may result in total systemic drug delivered over time (as measured by AUC) of 500 μg-hr/ml. In coadministration (i.e., in the presence of the bioenhancer) the systemic drug AUC will increase to 700 μg-hr/ml. However, if significantly increased drug bioavailability in the presence of the bioenhancer is anticipated, drug doses may need to be reduced for safety. Systemic drug concentrations are measured using standard in vitro or in vivo drug measurement techniques. “Systemic drug concentration” refers to a drug concentration in a mammal's bodily fluids, such as serum, plasma or blood; the term also includes drug concentrations in tissues bathed by the systemic fluids, including the skin. Systemic drug concentration does not refer to digestive fluids. The increase in total systemic drug concentrations is one way of defining an increase of drug bioavailability due to coadministration of bioenhancer and drug. For drugs excreted unmetabolized in the urine, an increased amount of unchanged drug in the urine will reflect the increase in systemic concentrations.

Increased Drug Bioavailability by Inhibition of P-gp

One embodiment of the present invention further increases bioavailability by increasing net drug absorption in the gut. Traditionally, drug absorption by the gut was considered to be the result of a passive diffusion process. Drugs were thought to diffuse into the gut based on the concentration gradient across the gut epithelial cells. Net drug transport across the gut, however, is the net result of drug influx and back flux, some of which is active drug transport. Drug influx is the flux from lumen to blood. Drug back flux is from blood or epithelium cytoplasm into the lumen. The invention reduces P-gp active drug transport across the luminal membrane to prevent return of drugs absorbed into the cytoplasm of the enterocytes back to the lumen of the gut.

Generally, practice of the method will reduce P-gp active drug transport in order to increase the net transport of drugs across the gut epithelium. An epithelium exists in a number of different tissue types including, but not limited to, the epithelia of the skin, liver, kidneys, adrenals, intestine, and colon. Such epithelia would be affected by systemic administration of P-gp inhibitors. However, the major effects of the invention will be limited to the gut because of concentration effects resulting from oral delivery.

Because of the many different compounds of Formula I that can act as inhibitors, the oral dosage of inhibitor to be present in the formulation (or elsewise as described below) is best determined empirically, as the dosage will depend on the affinity of the inhibitor for P-gp relative to the drug's affinity for P-gp. There are a number of assays available that allow the desired dosage to be readily determined without requiring clinical trials. While the actual dosage of inhibitor in a clinical formulation might be optimized from this initial dosage depending on results of a clinical trial, the assay as described is sufficient to establish a utilitarian dosage level.

Treatment of Tumors by Inhibition of P-gp

Tumors may be resistant to chemotherapy through the activity of P-glycoprotein. This may occur prior to or subsequent to treatment with anti-cancer chemotherapeutics. Formulations comprised of anti-cancer therapeutic compounds with bioenhancers of Formula I will have increased activity when administered to P-glycoprotein-expressing tumors which are otherwise resistant to the anti-cancer therapeutic agent if administered alone.

The invention therefore includes a method of treating a tumor, the method comprising coadministering to a mammal in need thereof a therapeutically effective amount of a compound of Formula I and a therapeutic agent, especially a chemotherapeutic agent. The coadministration can take place via the creation of a single composition. So a method of treating a tumor, the method comprising administering to a mammal in need thereof a therapeutically effective amount of a composition, the composition including a therapeutic agent such as a chemotherapeutic agent and a compound of Formula I, is part of the invention. The tumor treated by this method may be drug-resistant or it may have converted from drug-sensitive to drug-resistant. The therapeutic agent used according to this method is a P-glycoprotein substrate. For example, it may be selected from doxorubicin, vinblastine, vincristine, epipodophyllotoxin, taxanes, paclitaxel, docetaxel, etoposide, tenopiside, colchicines, daunorubicin, topotecan, actinomycin D, mitoxantrone, mitomycin C.

A related aspect of the invention is a method of preventing multidrug resistance in tumor cells, the method comprising administering an effective amount of a compound of Formula I to the tumor cells. The invention is also a method for increasing the sensitivity of tumor cells that have converted from sensitivity to chemotherapeutic agents to resistance to the chemotherapeutic agents. Specifically, the method comprises coadministering to a mammal a therapeutically effective amount of a compound of Formula I and at least one of the chemotherapeutic agents. Also included as part of the invention is a pharmaceutical composition for increasing the sensitivity of tumor cells that have converted from sensitivity to chemotherapeutic agents to resistance to the chemotherapeutic agents. The pharmaceutical composition comprises

(1) a therapeutically effective amount of a compound of Formula I, (2) at least one of the chemotherapeutic agents, and (3) a pharmaceutically acceptable carrier.

Delivery of Therapeutic Agents to the Central Nervous System by Inhibition of P-qlycoprotein

Recent data have demonstrated that P-glycoprotein is an integral part of the blood-brain barrier where it functions to prevent entry of drugs into the brain. Experiments with genetically altered mice which lacks P-glycoprotein demonstrate increased access of drugs such as anti-cancer agents, anti-HIV protease inhibitors, cardiac glycosides agents and sedatives into the brain. Thus inhibition of P-glycoprotein by the compounds described in Formula I is anticipated to increase the absorption of all pharmaceutical compounds which are P-glycoprotein substrates into the central nervous system.

Thus, another aspect of the invention is a method of delivery of pharmaceutical agents to the central nervous system. More specifically, the invention includes a method of delivering a pharmaceutical compound to the central nervous system of a patient, the method comprising coadministering the pharmaceutical compound with a compound of Formula I.

Oral Formulations

Another by-product of discovering agents for P-gp inhibition is that they may provide a useful means of creating a novel oral formulation. The oral formulation includes a bioenhancer, specifically a P-gp inhibitor plus a pharmaceutical compound. The pharmaceutical compound may have been previously administered by some non-oral delivery means to the patient. The combination of the P-gp inhibitor with the drug results in an improved formulation.

The invention therefore includes a method of converting a non-orally bioavailable pharmaceutical composition into an orally bioavailable pharmaceutical composition and also includes the composition formed by this means. Specifically, the method comprises formulating a composition including a compound of Formula I and the non-orally bioavailable pharmaceutical composition and optionally a pharmaceutically acceptable carrier.

In vitro P-gp Assays for Bioavailability

### Everted Gut Assays

Everted intestine can be prepared by methods known in the art (Hsing et al., Gastroenterology, 102:879-85 (1992)). In these studies rat small intestines turned “inside out” (i.e. the mucosal (or luminal) surface turned outside and the serosal surface inside) are bathed in a drug containing solution with and without the addition of the bioenhancer. The serosal surface of the small intestine is bathed in a solution that is periodically monitored or changed for the purpose of drug or bioenhancer measurement. For instance the everted rat small intestines can be bathed in a physiological saline solution loaded with Rhodamine 123 (Rh123) and the flux of Rh 123 monitored into the serosal solution. The addition of a bioenhancer in this set-up will increase Rh 123 transport into the serosal solution. An increase in drug or Rh 123bioavailability will be determined as follows:

$$\frac{X}{Y} ( 100 )$$

where Y is the initial rate of Rh 123 transport, and X is the initial rate of rhodamine transport in the presence of a bioenhancer. The initial rates will be determined as a linear relationship between time and Rh 123 concentration in the luminal solution. Alternatively, the serosal side of rat small intestines is bathed with the drug or bioenhancer of interest and the mucosal solution is monitored, as described in Hsing et al. (1992).

### Selection of a P-gp Inhibitor Based on Cell Growth Assays

This assay will be used to select candidate bioenhancers. Cells cultured with cytotoxic agents that are known P-gp transport substrates will be grown as controls in the absence of either drug or bioenhancer. The appKᵢ (apparent inhibition constant) for cell growth by drugs will be determined by varying the drug concentration in the culture medium. The appKᵢ will be expressed as the concentration of drug required to produce 50% inhibition of cell growth. Cells will also be grown in the presence of drug and bioenhancer. The bioenhancer will act to shift the appKᵢ to lower drug concentrations necessary for inhibition of cell growth. Cells with MDR can be used in this assay as described in Hait, W. N., et al., Biochemical Pharmacology 1993, 45:401-406. The method sections of Hait, W. N., et al. (1993) are herein incorporated by reference. Preferred bioenhancers will decrease the appKᵢ for a drug by at least 2 times, more preferably by at least 3 times, and even more preferably by at least 6 times.

### Rhodamine (Rh 123) Cellular Assay of P-gp Drug Transport and Drug Bioavailability

Rh 123 can be used in a cellular assay to monitor the bioavailability of drugs. Rh 123 transported by P-gp in this system acts as a drug, where P-gp pumps the Rh 123 out of the cell. Single cells or a population of cells can be monitored for the Rh 123 fluorescence which is indicative of P-gp transport. The cell types used will contain a P-gp transporter from an MDR strain such as those listed in Nielsen and Skovsgaard, Biochimica et Biophysica Acta, 1139:169-183 (1993) and herein incorporated by reference. Cells are loaded with Rh 123 in the presence of 15 nanograms per ml to 500 nanograms per ml of Rh 123 in a physiologically compatible buffer such as 3-N-morpholinopropanesulfonic acid (MOPS) with the suitable concentrations of sodium, potassium, and calcium chloride and an energy source. The cells are loaded with Rh 123 for 30-60 minutes depending on the temperature (37° C. or room temperature). The loaded cells are then washed and resuspended in buffer free of Rh 123. The efflux of Rh 123 can be determined using a fluorimeter. In the absence of any bioenhancer Rh 123 will be pumped out of the cell due to the action of P-gp, leading to a reduced amount of Rh 123 fluorescence from the cell.

Addition of a P-gp substrate or inhibitor either by preincubation after the cells have been washed with Rh 123 free buffer or during the efflux of Rh 123 from the cell will cause retention of Rh 123 within the cell. Retention of Rh 123 in the cell will be caused by the addition of a bioenhancer. Increased drug bioavailability is defined as the increase in Rh 123 retention within the cell. Compounds that increase Rh 123 retention are useful as bioenhancers.

Rh 123 retention in the absence of a bioenhancer will be determined by total Rh 123 cell fluorescence minus background Rh 123 cell fluorescence. An increase in drug bioavailability due to the addition of the bioenhancer will be the percentage increase in Rh 123 fluorescence retention as described by:

$$\frac{X}{Y} ( 100 )$$

where X equals Rh 123 fluorescence in the presence of the bioenhancer minus the background Rh 123 fluorescence and Y equals the Rh 123 fluorescence in the absence of the bioenhancer minus the background Rh 123 fluorescence.

The background Rh 123 fluorescence can be measured in a variety of ways including, but not limited to, the residual amount of Rh 123 fluorescence at the end of the experiment, the residual amount of Rh 123 fluorescence remaining based on an extrapolation of first order rate kinetics describing the efflux of Rh 123 from the cell, the residual amount of Rh 123 fluorescence in the presence of a sufficient amount of membrane detergents such as triton or digitonin, or the amount of Rh 123 fluorescence in the presence of a potassium-valinomycin clamp.

The addition of both a second drug and a bioenhancer to the Rh 123 assay will not necessarily cause an increased amount of Rh 123 retention compared to the presence of either the bioenhancer alone or the second drug alone. This is because Rh 123 retention can already be very high due to the second drug or bioenhancer concentration. Extra retention due to the addition of either the second drug or the bioenhancer can be difficult to measure above the signal for Rh 123 in the presence of the second drug or bioenhancer alone. However, once it has been determined that the drug (or second drug alone) increases Rh 123 fluorescence, i.e. decreases Rh 123 efflux, it can be assumed that the drug (or second drug alone) is transported by the P-gp transport system.

Vesicle Assays of P-gp Activity and Drug Bioavailability

A particularly preferred assay uses brush border membranes. Brush border membrane vesicles are prepared from the small intestine by methods known in the art, such as Hsing, S. et al, Gastroenterology 102:879-885 (1992). The vesicles will be assayed for the presence of P-gp by using monoclonal antibodies directed to P-gp either using SDS page gel electrophoresis and western blotting techniques or using immunochemistry and electromicroscopy. Vesicles containing P-gp will be used for drug transport assays.

Drug transport assays consist of measuring the transport of drugs into the vesicles in an adenosine triphosphate (ATP) dependent fashion. Uptake of the drug in the presence of ATP will be monitored using fluorescence or absorbance techniques, for instance using Rh 123 as the fluorescent drug transported into the interior of the vesicle. Radioactively labeled drugs can also be used to monitor drug transport into the interior of the vesicle using a filter wash system. The addition of ATP will induce the transport of the drug into the vesicle and will increase drug transport compared to passive diffusion of the drug into the vesicle interior. Addition of non-hydrolyzable analogs of ATP such as ATP gamma S or adenosine monophosphate para-nitrophenol (AMP-PNP) will not produce an ATP dependent influx of drug into the vesicle. Thus, the introduction of a non-hydrolyzable nucleotide can be used as a control to monitor whether drug transport has actually occurred due to ATP hydrolysis from the P-gp transport system.

The addition of a bioenhancer to this assay system using a fluorescent drug or a radioactive drug and monitoring its uptake, will reduce the uptake of the drug into the interior of the vesicle with the addition of ATP. This reduction in drug transport represents an increase of the bioavailability of the drug. The vesicles transporting drugs in an ATP dependent fashion are oriented with the cystolic face of the P-gp accessible to the ATP. It is these vesicles that hydrolyze the ATP and transport the drug into the interior of the vesicle. The interior of the vesicle in turn corresponds to the luminal surface or the apical membrane of the brush border cells. Thus, transport into the lumen of the vesicle or interior of the vesicle corresponds to transport into the lumen of the gut. A decrease in the transport of the lumen of the vesicle is the equivalent of increasing net drug absorption and increasing the drug bioavailability.

### P-gp ATPase Assays of P-gp Activity and Drug Bioavailability

P-gp molecules can be isolated in vesicles suitable for measuring ATPase activity. P-gp ATPase activity will be measured in the presence of other types of ATPase inhibitors, such as, but not limited to, sodium potassium ATPase inhibitors (ouabain and vanadate), mitochondrial ATPase inhibitors such as oligomycin, and alkaline phosphatase inhibitors. The ATPase assays will also be conducted in the absence of sodium and potassium to eliminate background sodium and potassium ATPase activity. ATPase activity will be measured as ATPase activity dependent on the presence of a drug such as daunomycin. ATPase activity will be measured using ATP or hydrolyzable ATP analogs such para-nitrophenolphosphate. The production of product will be monitored using phosphate assay procedures of those of Yoda, A. and Hokin, L., Biochem. Biophys. Res. Comm., 40:880-886 (1970) or by monitoring phosphatase activity as recognized in the literature.

An increase in P-gp ATPase activity due to the addition of a drug is recognized as an increase in drug bioavailability. P-gp molecules located in the brush border membrane vesicles are oriented so the cytosolic portion of the molecule finds and hydrolyzes ATP. It is these P-gp molecules that will give rise to the drug dependent ATPase activity. Bioenhancer that is able to stimulate the ATPase activity will be able to compete with the drug for the P-gp transport system. Such bioenhancers will decrease P-gp drug transport due to their increased ability to stimulate P-gp activity. Bioenhancers can also inhibit drug dependent P-gp ATPase activity without stimulating P-gp ATPase activity thus, inhibiting drug transport.

Another manner of determining the amount of bioenhancer appropriate for an oral formulation is based on the Kᵢ of the specific inhibitor (for whichever binding is being measured). An appropriate amount of inhibitor is one that is sufficient to produce a concentration of the bioenhancer in the lumen of the gut of the animal of at least 0.1 times the Kᵢ of the bioenhancer.

In all of these cases, the goal of selecting a particular concentration is increased bioavailability of the pharmaceutical compound that is being administered. Thus, a desirable goal is to provide integrated systemic concentrations over time of the pharmaceutical compound in the presence of the inhibitor that is greater than the integrated systemic concentrations over time of the pharmaceutical compound in the absence of the inhibitor by at least 10% of the difference between bioavailability in its absence and complete oral bioavailability. Preferred is attaining of “complete bioavailability,” which is 100% systemic bioavailability of the administered dosage.

Screening Assay for Bioenhancers

In summary, the various techniques described above for screening candidate bioenhancer compounds for activity by assaying for inhibition in the gut of a mammal of transport by P glycoprotein are all generally useful as methods of identifying compounds that are useful for increasing bioavailability of a drug in a mammal. In all of these assays, the best bioenhancers are those compounds selected from the candidate compounds being tested that best inhibit transport of a tested drug in the gut of the mammal (either by direct testing in vivo or by a test that predicts such activity). When testing for inhibition of activity of a cytochrome enzyme, assays that detect inhibition of P-gp-mediated-transport (for a particular mammal, particularly human) are preferred. Although in vivo assays are preferred, because of the direct relationship between the measurement and gut activity, other assays, such as assays for inhibition of P-gp-mediated-transport in isolated enterocytes or microsomes obtained from enterocytes of the mammal in question or for inhibition of P-gp-mediated-transport in a tissue or membrane from the gut of said mammal, are still useful as screening assays.

Coadministration and Delivery of Bioenhancers

### Increase in Drug Bioavailability with Coadministration of a Bioenhancer and a Drug

The present invention will increase the bioavailability of the drug in the systemic fluids or tissues by co-administering the bioenhancer of Formula I with a drug. “Coadministration” includes concurrent administration (administration of the bioenhancer and drug at the same time) and time-varied administration (administration of the bioenhancer at a time different from that of the drug), as long as both the bioenhancer and the drug are present in the gut lumen and/or membranes during at least partially overlapping times. Systemic fluids or tissues refer to drug concentration measured in blood, plasma or serum, and other body fluids or tissues in which drug measurements can be obtained.

### Delivery Vehicles Provide For Coadministration

Coadministration can vary in the type of delivery vehicle. The bioenhancer and the drug can use different delivery vehicles such as, but not limited to, time release matrices, time release coatings, companion ions, and successive oral administrations. Alternatively, the drug and the bioenhancer can be formulated with different coatings possessing different time constants of bioenhancer and drug release. The use of bioenhancers also applies to epithelia tissues other than the gut. Aspects of the invention used in the gut are appropriately used in other types of epithelia. For example, P-glycoprotein has also been demonstrated in the skin and bioenhancers used in transdermal formulations would increase drug bioavailability to systemic fluids and tissues. Such applications are included as part of the invention herein because of inhibition by bioenhancers of P-glycoprotein in epithelia other than the gut.

### Formulations of Bioenhancers

The invention is carried out in part by formulating an oral pharmaceutical composition to contain a bioenhancer of Formula I . This is accomplished in some embodiments by admixing a pharmaceutical compound, a pharmaceutical carrier, and a bioenhancer comprising an inhibitor of P-glycoprotein-mediated transport, the bioenhancer being present in sufficient amount to provide integrated systemic concentrations over time of the compound as measured by AUC's greater than the integrated systemic concentrations over time of the compound in the absence of the composition when the pharmaceutical composition is administered orally to an animal being treated with the pharmaceutical composition. A pharmaceutical carrier increases drug solubility or protects drug structure or aids in drug delivery or any combination thereof.

Pharmaceutical compositions produced by the process described herein are also part of the present invention.

In addition to use with new formulations, the present invention can also be used to increase the bioavailability of the active compound of an existing oral pharmaceutical composition. When practiced in this manner, the invention is carried out by reformulating the existing composition to provide a reformulated composition by admixing the active compound with a bioenhancer of Formula 1, the bioenhancer being present in sufficient amount to provide integrated systemic concentrations over time of the compound when administered in the reformulated composition greater than the integrated systemic concentrations over time of the compound when administered in the existing pharmaceutical composition. All of the criteria described for new formulations also apply to reformulation of old compositions. In preferred aspects of reformulations, the reformulated composition comprises all components present in the existing pharmaceutical composition plus the bioenhancer, thus simplifying practice of the invention, although it is also possible to eliminate existing components of formulations because of the increase in bioavailability. Thus, the invention also covers reformulated compositions that contain less than all components present in the existing pharmaceutical composition plus the bioenhancer. However, this invention does not cover already existing compositions that contain a component which increases bioavailability by mechanisms described in this specification (without knowledge of the mechanisms), should such compositions exist.

Traditional formulations can be used with the bioenhancer compounds of Formula I. Optimal bioenhancer doses can be determined by varying the coadministration of bioenhancer and drug in time and amount dependent fashion and monitoring bioavailability. Once the optimal bioenhancer dose is established for a drug the formulation (bioenhancer, drug and formulation composition(s)) is tested to verify the increased bioavailability. In the case of time or sustained release formulations it will be preferred to establish the optimal bioenhancer dose using such formulations from the start of the bioavailability experiments.

Dosage levels of the order of from about 0.001 mg to about 100 mg of a compound of Formula I per kilogram body weight per day are useful in the present invention. The amount of active ingredient, which is herein defined as a compound of Formula I present as a counterion of a pharmaceutical drug, that may be combined with carrier materials to produce a single dosage form will vary depending upon the host treated and the particular mode of administration. Dosage unit forms will generally contain between about 1 mg to about 500 mg of an active ingredient.

The specific dose level for any particular individual will depend upon a variety of factors including the activity of the compound of Formula I, the age, body weight, general physical and mental health, genetic factors, environmental influences, sex, diet, time of administration, route of administration, rate of excretion, and the severity of the particular problem being treated. For example, the dose level useful for increasing drug availability of a co-administered drug may vary among individuals depending on the oral bioavailability of the co-administered drug. Similarly, the dose level for treating MDR resistance in cancer cells may vary among individuals, depending upon the severity of the individual's symptoms.

While it is possible for an active bioenhancer of Formula I to be administered alone, it is preferable to present it as a formulation. Formulations of the present invention suitable for oral administration may be in the form of discrete units such as capsules, cachets, tablets, or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. The active ingredient may also be in the form of a bolus, electuary, or paste.

A tablet may be made by compressing or molding the active ingredient optionally with one or more accessory ingredients. Compressed tablets may be prepared by compressing, in a suitable machine, the active ingredient in a free-flowing form such as a powder or granules, optionally mixed with a binder, lubricant, inert diluent, surface active, or dispensing agent. Molded tablets may be made by molding, in a suitable machine, a mixture of the powdered active in ingredient and a suitable carrier moistened with an inert liquid diluent.

The formulations, for human medical use, of the present invention comprise an active ingredient in association with a pharmaceutically acceptable carrier therefor and optionally other therapeutic ingredient(s). The carrier(s) must be “acceptable” in the sense of being compatible with the other ingredients of the formulations and not deleterious to the recipient thereof. The bioenhancer of Formula I is preferably present as a counter ion of the pharmaceutical compound in order to ensure that the bioenhancer is present at maximum concentration in the presence of the drug that it is protecting.

The pharmacologically active compounds of the invention are useful in the manufacture of pharmaceutical compositions comprising an effective amount thereof in conjunction or admixture with the excipients or carriers suitable for either enteral or parenteral application. Preferred are tablets and gelatin capsules comprising the active ingredient together with one or more of the following: (a) diluents, such as lactose, dextrose, sucrose, mannitol, sorbitol, cellulose, glycine and the like; (b) lubricants, such as silica, talcum, stearic acid, its magnesium or calcium salt, polyethyleneglycol and the like; for tablets also; (c) binders, such as magnesium aluminum silicate, starch paste, gelatin, tragacanth, methylcellulose, sodium carboxymethyl-cellulose or polyvinylpyrrolidone and the like; and, if desired, (d) disintegrants, such as effervescent mixtures and the like; and (e) absorbents, colorants, flavors, and sweeteners and the like. In addition, they may also contain other therapeutically valuable substances. Said compositions are prepared according to conventional mixing, granulating, or coating methods, respectively, and contain about 0.1 to 75%, preferably about 1 to 50%, of the active ingredient.

The formulations may conveniently be presented in unit dosage form and may be prepared by any of the methods well known in the art of pharmacy. All methods include the step of bringing the active ingredient into association with the carrier that constitutes one or more accessory ingredients. In general, the formulations are prepared by uniformly and intimately bringing the active ingredient into association with a liquid carrier or a finely divided solid carrier or both, and then, if necessary, shaping the product into the desired formulation.

### EXAMPLES

### Example 1

### 2-[2-(4-Benzylpiperidine-1-carbonyl)-3-(3,4-dimethoxyphenyl)-5,6-dimethoxy-indan-1-yl]-1-(4-benzylpiperidin-1-yl)-ethanone

### AV-159

A solution of 1-carboxymethyl-3-(3,4-dimethoxyphenyl)-5,6-dimethoxy-indan-2-carboxylic acid (0.2 g, 0.48 mmol), 1-ethyl-3-(3-dimethylaminopropyl)carbodiimide hydrochloride (0.36 g, 1.92 mmol), triethylamine (0.26 ml, 1.92 mmol) and dimethylaminopyridine (17 mg, 0.144 mmol) in 10 ml of THF was prepared. The resulting mixture was stirred under argon at room temperature for 3 hours. Benzylpiperazine (0.17g, 1.92 mmol) was added to the mixture. The resulting mixture was stirred overnight at room temperature. The white precipitate was filtered. The crude filtered solution was washed with a saturated solution of sodium bicarbonate. The aqueous layer was extracted with dichloromethane and the combined organic layers were dried over MgSO₄ and evaporated. Flash chromatography of the residue over silica gel using an ethyl acetate/hexane gradient gave 130 mg of a white oily solid in a 37% yield.

¹H NMR (300 MHz, CDCl₃) δ 0.6-1.8 (m, 1OH), 2.1 (m, 3H), 2.5 (m, 8H), 2.9 (m, 1H), 3.5

(m, 1H), 3.6-3.8 (m, 12H), 4 (m, 1H), 4.6 (m, 2H), 5 (d, 1H), 6.4 (t, 1H), 6.8 (m, 4H), 7.2

(m, 10H). ESIMS,m/z for C₄₆H₅₄N₂O₆ [M+Na]⁺:753

### Example 2

### 2-[3-(3,4-Dimethoxyphenyl)-5,6-dimethoxy-2-(4-phenylpiperazin-1-carbonyl)-indan-1-yl]-1-(4-phenylpiperazin-1-yl)-ethanone

### AV-161

Prepared analogously to Example 1 from 1-phenylpiperazine in a yield of 27%

(90 mg), a white powder was obtained.

ESIMS,m/z for C₄₂H₄₈N₄O₆ [M+H]⁺:705.3 [M+Na]+: 728.3

### Example 3

### 2-[2-[4-(4-Acetylphenyl)-piperazin-1-carbonyl]-3-(3,4-dimethoxyphenyl)-5,6-dimethoxy-indan-1-yl]-1-[4-(4-acetylphenyl)piperazine-1-yl]-ethanone

### AV-163

Prepared analogously to Example 1 from 1-(4-piperazin-1-yl-phenyl)-ethanone in a yield of 24% (91 mg), light yellow crystals were obtained.

ESIMS, m/z for C₄₆H₅₂N₄O₈ [M+H]⁺:789.4 [M+Na]+: 812.4

### Example 4

### 2-[2-(4-Benzyl-piperazine-1-carbonyl)-3-(3,4-dimethoxyphenyl)-5,6-dimethoxy-indan-1-yl]-1-(4-benzylpiperazine-1-yl)-ethanone

### AV-164

Prepared analogously to Example 1 from 1-benzylpiperazine in a yield of 5% (17 mg), a fluffy white powder was obtained.

ESIMS, m/z for C₄₄H₅₂N₄O₆ [M+H]⁺:732.918

### Example 5

### 2-[2-[4-(4-Chlorophenyl)-piperazin-1-carbonyl]-3-(3,4-dimethoxyphenyl)-5,6-dimethoxy-indan-1-yl]-1-[4-(4-chlorophenyl)piperazine-1-yl]-ethanone

### AV-167

Prepared analogously to Example 1 from 1-(4-chlorophenyl)-piperazine in a yield of 16% (61 mg), a white powder was obtained.

ESIMS, m/z for C₄₂H₄₆N₄O₆C₁₂ [M+2H]⁺:775.2

### Example 6

### 2-{3-(3,4-Dimethoxyphenyl)-5,6-dimethoxy-2-[4-(4-methoxyphenyl)-piperazin-1-carbonyl]-indan-1-yl)-1-[4-(4-methoxyphenyl)-1-yl]-ethanone

### AV-168

Prepared analogously to Example 1 from 1-(4-methoxyphenyl)-piperzine in a yield of 24% (88 mg), light brown crystals were obtained.

ESIMS, m/z for C₄₄H₅₂N₄O₈ [M+H]⁺:765.4

### Example 7

### 2-(2-[4-(4-Fluorophenyl)-piperazin-1-carbonyl]-3-(3,4-dimethoxyphenyl)-5,6-dimethoxy-indan-1-yl}-1-[4-(4-fluorophenyl)piperazine-1-yl]-ethanone

### AV-169

Prepared analogously to Example 1 from 1-(4-fluorophenyl)-piperazine in a yield of 18% (63 mg), white crystals were obtained.

ESIMS, m/z for C₄₂H₄₆N₄O₆F₂ [M+H]⁺:741.3

### Example 8

### 2-{3-(3,4-Dimethoxyphenyl)-5,6-dimethoxy-2-(pyrimidin-2-y-piperazin-1-carbonyl)-indan-1-yl]-1-(pyrimidin-2-yl-piperazine-1-yl)-ethanone

### AV-170

Prepared analogously to Example 1 from 2-piperazin-1-yl-pyrimidine in a yield of 12% (45 mg), white crystals were obtained.

ESIMS, m/z for C₃₈H₄₄N₈O₆ [M+H]⁺:709.3

### Example 9

### 2-{3-(3,4-Dimethoxyphenyl)-5,6-dimethoxy-2-[4-(2-ethoxyphenyl)-piperazin-1-carbonyl]-indan-1-yl}-1-[4-(2-ethoxyphenyl)-1-yl]-ethanone

### AV-171

A solution of 1-carboxymethyl-3-(3,4-dimethoxyphenyl)-5,6-dimethoxy-indan-2-carboxylic acid (0.2 g, 0.48 mmol), 1-ethyl-3-(3-dimethylaminopropyl)carbodiimide hydrochloride (0.36 g, 1.92 mmol), triethylamine (0.26 ml, 1.92 mmol) and dimethylaminopyridine (17 mg, 0.144 mmol) in 10 ml of THF was prepared. The resulting mixture was stirred under argon at room temperature for 3 hours. 1-(2-ethoxyphenyl)piperazine hydrochloride (0.466 g, 1.92 mmol) was added to the mixture. The resulting mixture was stirred overnight at room temperature. The white precipitate was filtered. The crude filtered solution was washed with a saturated solution of sodium bicarbonate. The aqueous layer was extracted with dichloromethane and the combined organic layers were dried over MgSO₄ and evaporated. Flash chromatography of the residue over silica gel using an ethyl acetate/hexane gradient gave 161 mg of a white crystalline solid in a 42% yield.

The enantiomeric separation was performed using a Chiralceirod™ 10×50 cm column, with an eluent of MeOH, at 30° C. with detection of the eluted fractions at UV 240 nm. Peak 1 was at time 7.660 minutes with an enantiomeric excess (% e.e.) of 99.1. Peak 2 was at time 8.981 minutes with an % e.e. of 86.3. Optical rotation of AV-171 peak 1 and AV-171 peak 2 was measured with a Perkin Elmer 241 polarimeter at a wavelength of 589 nm using a 5 second integration time and a 10 cm, 1 ml water-jacketed cell filled at +25 and −25, at 0.5° C., which gave a specific rotation for peak 1 of +95.7° C. and for peak 2 of −94.1° C.

¹H NMR (300 MHz, CDCl₃) δ 1.29 (t, 3H), 2.21 (t, 3H), 2.2 (dd, 1H), 2.98 (m,1H), 3.2-3.4 (m, 8H), 3.4-3.55 (m, 2H), 3.7 (s, 3H), 3.8 (s, 3H), 3.9 (s, 6H), 3.3-4.0 (m, 8H), 4.9 (d, 2H), 6.4 (s, 1H), 6.7-7.0 (m, 7H), 7.2-7.4 (m, 5H). ESIMS, m/z for C₄₆H₅₆N₄O₈ [M+H]⁺:793.4

### Example 10

### 2-{3-(3,4-Dimethoxyphenyl)-5,6-dimethoxy-2-[4-(3-phenylallyl)-piperazine-1-carbonyl]-indan-1-yl}-1-[4-(3-phenylallyl)piperazine-1-yl]-ethanone

### AV-172

Prepared analogously to Example 1 from 1-(3-phenylallyl)-piperazine in a yield of 8% (31 mg), a white powder was obtained. ESIMS, m/z for C₄₈H₅₆N₄O₆ [M+H]⁺:785.4

### Example 11

### 1-[(Benzylethylcarbamoyl)-methyl]-3-(3,4-dimethoxyphenyl)-5,6-dimethoxy-indan-2-carboxylic acid benzyl ethyl amide

### AV-173

Prepared analogously to Example 1 from benzylethylamine in a yield of 9% (28 mg), a white powder was obtained.

ESIMS, m/z for C₄₀H₄₆N₂O₆ [M+H]⁺:651.4, [M+Na]⁺:673.3

### Example 12

### 4-{2-[3-(3,4-Dimethoxyphenyl)-5,6-dimethoxy-2-(piperazine-1-carboxylic acid ethyl ester)-indan-1-yl]-acetyl}-piperazine-1-carboxylic acid ethyl ester

### AV-174

Prepared analogously to Example 1 from piperazine-1-carboxylic acid ethyl ester in a yield of 20% (66 mg), white crystals were obtained.

ESIMS, m/z for C₃₆H₄₈N₄O₁₀ [M+H]⁺:697.4,[M+Na]⁺:719.3

### Example 13

### 4-{2-[3-(3,4-Dimethoxyphenyl)-5,6-dimethoxy-2-(piperazine-1-carboxylic acid benzyl ester)-indan-1-yl]-acetyl}-piperazine-1-carboxylic acid benzyl ester

### AV-179

Prepared analogously to Example 1 from piperazine-1-carboxylic acid benzyl ester in a yield of 14% (56 mg), transparent white crystals were obtained.

ESIMS, m/z for C₄₆H₅₂N₄O₁₀ [M+H]⁺:821.4, [M+Na]⁺:844.3

### Example 14

### 4-{2-[3-(3,4-Dimethoxyphenyl)-5,6-dimethoxy-2-(piperazine-1-carboxylic acid t-butyl ester)-indan-1-yl]-acetyl}-piperazine-1-carboxylic acid t-butyl ester

### AV-180

Prepared analogously to Example 1 from piperazine-1-carboxylic acid t-butyl ester, the product was obtained in a yield of 15% (54 mg).

ESIMS, m/z for C₄₀H₅₆N₄O₁₀ [M+H]⁺:753.4

### Example 15

### 1-(3,4-Dimethoxyphenyl)-3-dimethylcarbamoylmethyl-5,6-dimethoxy-indan-2-carboxylic acid dimethyl amide

### AV-181

Prepared analogously to Example 1 from dimethylamine in a yield of 3% (6 mg), white crystals were obtained.

ESIMS, m/z for C₂₆H₃₄N₂O₆ [M+H]⁺:471.3, [M+Na]⁺:493.2

### Example 16

### 1-(3,4-Dimethoxyphenyl)-3-diethylcarbamoylmethyl-5,6-dimethoxy-indan-2-carboxylic acid diethyl amide

### AV-182

Prepared analogously to Example 1 from diethylamine the product was obtained in a yield of 5% (12 mg).

ESIMS, m/z for C₃₀H₄₂N₂O₆ [M+H]⁺:527.3,[M+Na]⁺:549.3

### Example 17

### 2-[3-(3,4-Dimethoxyphenyl)-5,6-dimethoxy-2-(piperazine-1-carbonyl)-indan-1-yl]-1-piperazin-1-yl-ethanone

### AV-185

Prepared analogously to Example 1 from piperazine-1-carboxylic acid t-butyl ester, except that the additional step of deprotection of the t-BOC group using dichloromethane and TFA was carried out according to the method of Green et al., in Protective Groups in Organic Synthesis, 3rd ed., 1999, pp. 518-522. Off-white crystals were obtained in a yield of 100% (33 mg).

ESIMS, m/z for C₃₀H₄₀N₄O₆ [M+H]⁺:553.3

### Example 18

### 2-[3-(3,4-Dimethoxyphenyl)-5,6-dimethoxy-2-(4-ethylpiperazine-1-carbonyl)-indan-1-yl]-1-(4-ethylpiperazin-1-yl)-ethanone

### AV-187

Prepared analogously to Example 1 from 1-ethylpiperazine in a yield of 14% (13 mg), a thick, clear oil was obtained.

ESIMS, m/z for C₃₄H₄₈N₄O₆ [M+H]⁺:609.4

### Example 19

### 2-γ3-(3,4-Dimethoxyphenyl)-5,6-dimethoxy-2-[4-(2-methoxyphenyl)-piperazin-1-carbonyl]-indan-1-yl}-1-[4-(2-methoxyphenyl)-1-yl]-ethanone

### AV-188

Prepared analogously to Example 1 from 1-(2-methoxyphenyl)-piperazine in a yield of 7% (26 mg), a fluffy white powder was obtained.

ESIMS, m/z for C₄₄H₅₂N₄O₈ [M+H]⁺:765.4

### Example 20

### 1-(3,4-Dimethoxyphenyl)-3-dipropylcarbamoylmethyl-5,6-dimethoxy-indan-2-carboxylic acid dipropyl amide

### AV-189

Prepared analogously to Example 1 from dipropylamine in a yield of 6% (18 mg), a white oily solid was obtained.

ESIMS, m/z for C₃₄H₅₀N₂O₆ [M+H]⁺:583.4

### Example 21

### 1-(3,4-Dimethoxyphenyl)-3-dibutylcarbamoylmethyl-5,6-dimethoxy-indan-2-carboxylic acid dibutyl amide

### AV-190

Prepared analogously to Example 1 from dibutylamine a clear oil was obtained.

ESIMS, m/z for C₃₈H₅₈N₂O₆ [M+H]⁺:639.4,[M+Na]⁺:661.4

### Example 22

### 1-[(Diisobutylcarbamoyl)methyl]-3-(3,4-dimethoxyphenyl)-5,6-dimethoxy-indan-2-carboxylic acid diisobutylamide

### AV-191

Prepared analogously to Example 1 from diisobutylamine in a yield of 10% (30 mg), white crystals were obtained. ESIMS, m/z for C₃₈H₅₈N₂O₆ [M+H]⁺:639.4, [M+Na]⁺:661.4

### Example 23

### 1{[Bis(2-ethylhexyl)carbamoyl]methyl}-3-(3,4-dimethoxyphenyl)-5,6-dimethoxy-indan-2-carboxylic acid bis(2-ethylhexyl) amide

### AV-192

Prepared analogously to Example 1 from bis-(2-ethylhexyl)amine in a yield of 168 mg (40%), a clear liquid was obtained.

ESIMS, m/z for C₅₄H₉₀N₂O₆ [M+H]⁺:863.3

### Example 24

### 1-(3,4-Dimethoxyphenyl)-3-dihexylcarbamoylmethyl-5,6-dimethoxy-indan-2-carboxylic acid dihexyl amide

### AV-193

Prepared analogously to Example 1 from dihexylamine in a yield of 5% (19 mg), a clear oil was obtained. ESIMS, m/z for C₄₆H₇₄N₂O₆ [M+H]⁺:751.6

### Example 25

### 2-{3-(3,4-Dimethoxyphenyl)-5,6-dimethoxy-2-(pyridin-2-y-piperazin-1-carbonyl)-indan-1-yl]-1-(pyridin-2-yl-piperazine-1-yl)-ethanone

### AV-195

Prepared analogously to Example 1 from 1-pyridin-2-yl-piperazine in a yield of 14% (49 mg), off-white crystals were obtained. ESIMS, m/z for C₄₀H₄₆N₆O₆ [M+H]⁺:706.84

### Example 26

### 2-{3-(3,4-Dimethoxyphenyl)-5,6-dimethoxy-2-[4-(4-nitrophenyl)-piperazin-1-carbonyl]-indan-1-yl}-1-[4-(4-nitrophenyl)-1-yl]-ethanone

### AV-196

Prepared analogously to Example 1 from 1-(4-nitrophenyl)-piperazine in a yield of 5% (18 mg), a bright yellow powder was obtained. ESIMS, m/z for C₃₆H₄₈N₄O₁₀ [M+H]⁺:707.3

### Example 27 2-[2-(4-Benzylpiperidin-1-yl)-methyl-3-(3,4-dimethoxyphenyl)-5,6-dimethoxy-indan-1-yl]-1-(4-benzylpiperidin-1-yl)-ethane

### AV-160

A solution of 160 mg, 0.217 mmol AV-159, 2-[2-(4-benzylpiperidine-1-carbonyl)-3-(3,4-dimethoxy-phenyl)-5,6-dimethoxy-indan-1-yl]-1-(4-benzylpiperidin-1-yl)-ethanone, in 10 ml anhydrous THF was combined with 0.87 ml (0.87 mmol) 1M lithium aluminum hydride solution in THF. The mixture was refluxed under argon for 6 h. The reaction mixture was cooled to room temperature. To this mixture was added 3 ml of 1 N sodium hydroxide. The mixture was stirred for 15 minutes, and the precipitated powder was filtered. To the mixture of the aqueous layer and the THF layer was added 5 ml EtOAc. The organic layer was separated, dried over MgSO₄, and evaporated. Purification of the residue by flash chromatography using an ethyl acetate/hexane gradient gave 110 mg

(72%) of a white powder.

¹H NMR (300 MHz, DMSO d6) δ 1-1.3 (m, 5H), 1.3-1.7 (m, 8H), 3.52 (s, 3H), 3.64 (s, 3H), 3.7 (s, 3H), 3.88 (d, 1H), 6.29 (s, 1H), 6.58 (dd, 1H), 6.81 (m, 2H), 7.06 (m, 2H), 7.13 (m, 4H), 7.23 (m, 4H); 13C NMR (300 MHz, DMSO d6) δ 25.02, 32.17, 32.31, 32.52, 37.79, 38.09, 39.32, 39.53, 39.73, 41.05, 42.88, 42.95, 50.6, 52.1, 52.3, 52.5, 54.8, 55.00, 55.02, 55.04, 55.06, 55.09, 56.00, 57.00, 108.79, 109.05, 112.15, 112.24, 120.52, 126.05, 128, 129.25, 129.33, 136.6, 137.9, 139.1, 140.6, 140.8, 147.5, 147.8, 148.00, 149.00.

### Example 28

### 2-[3-(3,4-Dimethoxyphenyl)-5,6-dimethoxy-2-(4-phenylpiperazin-1-yl) methyl-indan-1-yl]-1-(4-phenylpiperazin-1-yl)-ethane

### AV-162

Prepared analogously to Example 27 from AV-161,2-[2-(4-benzylpiperidin-1-yl)-methyl-3-(3,4-dimethoxyphenyl)-5,6-dimethoxy-indan-1-yl]-1-(4-benzylpiperidin-1-yl)-ethane in a yield of 98% (60 mg), a white powder was obtained.

ESIMS, m/z for C₄₂H₅₂N₄O₄ [M+H]⁺:676.89

### Example 29

### 2-{3-(3,4-Dimethoxyphenyl)-5,6-dimethoxy-2-[4-(4-methoxy)-piperazin-1-yl-methyl]-indan-1-yl}-1-[4-(4-methoxyphenyl)-piperazin-1-yl]-ethane

### AV-183

Prepared analogously to Example 27 from AV-168, 2-{3-(3,4-dimethoxyphenyl)-5,6-dimethoxy-2-[4-(4-methoxyphenyl)-piperazin-1-carbonyl]-indan-1-yl}-1-[4-(4-methoxyphenyl)-1-yl]-ethanone, in a yield of 42%, 20 mg were obtained.

ESIMS, m/z for C₄₄H₅₆N₄O₆ [M+H]⁺:737.5

### Example 30

### 2-{3-(3,4-Dimethoxyphenyl)-5,6-dimethoxy-2-[4-(2-ethoxyphenyl)-piperazin-1-yl-methyl]-indan-1-yl}-1-[4-(2-ethoxyphenyl)-piperazin-1-yl]-ethane

### AV-184

Prepared analogously to Example 27 from AV-171,2-{3-(3,4-dimethoxyphenyl)-5,6-dimethoxy-2-[4-(2-ethoxyphenyl)-piperazin-1-carbonyl]-indan-1-yl}-1-[4-(2-ethoxyphenyl)-1-yl]-ethanone, in a yield of 41% (20 mg), yellow brown crystals were obtained.

ESIMS, m/z for C₄₆H₆₀N₄O₆ [M+H]⁺:765.00

### Example 31

### 2-{3-(3,4-Dimethoxyphenyl)-5,6-dimethoxy-2-[4-(4-chlorophenyl)-piperazin-1-yl-methyl]-indan-1-yl}-1-[4-(4-chlorophenyl)-piperazin-1-yl]-ethane

### AV-186

Prepared analogously to Example 27 from AV-167, 2-[2-[4-(4-chlorophenyl)-piperazin-1-carbonyl]-3-(3,4-dimethoxyphenyl)-5,6-dimethoxy-indan-1-yl]-1-[4-(4-chlorophenyl)-piperazine-1-yl]-ethanone, in a yield of 52% (25 mg), off-white crystals were obtained.

ESIMS, m/z for C₄₂H₅₀Cl₂N₄O₄ [M+H]⁺:747.3

### Example 32

### [1-[2-t-Butyldiphenyl-silanoxy)-ethyl]-3-(3,4-dimethoxyphenyl)-5,6-dimethoxy-indan-2-yl]-methanol

### AV-175

A solution of 2-[3-(3,4-dimethoxyphenyl)-2-hydroxymethyl-5,6-dimethoxy-indane-1-yl]ethanol (0.2 g, 0.51 mmol) in 5 ml anhydrous dichloromethane was prepared. Under argon, imidazole (38.58 mg, 0.56 mmol) and t-butyldiphenylchlorosilane (0.153 g, 0.56 mmol) were added. Then the mixture was stirred overnight at room temperature. The resulting precipitate was filtered and the organic layer was extracted with saturated solution of sodium bicarbonate. The aqueous layer was washed three times with 10 ml of dichloromethane. The combined organic layer was dried over MgSO₄ and evaporated. Flash chromatography over silica using an ethyl acetate/hexane gradient gave 1.25 g (51%) of a white powder, AV-175, which is a compound of formula (7) of Scheme 3.

1H NMR (300 MHz, DMSO d6) δ 1(s, 9H), 1.6 (m, 4), 2 (m, 1H), 2.7 (m, 1H), 3.7 (s, 3H), 3.72 (m, 3H), 3.8 (s, 3H), 3.76-3.84 (m, 1H), 3.88 (s, 3H), 3.96 (d, 1H), 6.4 (s, 1H), 6.6-6.8 (m, 4H), 7.4 (m, 5H), 7.7 (m, 5H); ESIMS,m/z for C₃₈H46O6Si [M+Na]⁺:649.3

### Example 33

### t-Butyl-{2-[3-(3,4-dimethoxyphenyl)-5,6-dimethoxy-2-methoxymethyl-indan-1-yl]-ethoxy}-diphenylsilane

### Compound of Formula (8)

To the AV-175, [1-[2-t-butyldiphenyl-silanoxy)ethyl-3-(3,4-dimethoxyphenyl)-5,6-dimethoxy-indan-2-yl]-methanol, (1.2 g, 1.9 mmol) in 10 ml anhydrous dichloromethane, was added 2,6-di-t-butyl pyridine (2.2 g, 11.51 mmol) and methyl triflate (1.57 g, 9.59 mmol). The mixture was stirred at room temperature under argon overnight. To the mixture was added 50 ml of a saturated solution of sodium bicarbonate. Then the organic layer was dried over MgSO₄, filtered, and evaporated under vacuum to give 1.13 g (92%) of a white powder, t-butyl-{2-[3-(3,4-dimeithoxyphenyl)-5,6-dimethoxy-2-methoxymethyl-indan-1-yl]-ethoxy}-diphenylsilane, which is a compound of formula (8) of Scheme 3.

ESIMS, m/z for C39H48O6Si [M+Na]⁺:663.3

### Example 34

### 2-[3-(3,4-Dimethoxyphenyl)-5,6-dimethoxy-2-methoxymethyl-indan-1-yl]-ethanol

### AV-176

A compound of formula (8), t-butyl-{2-[3-(3,4-dimethoxyphenyl)-5,6-dimethoxy-2-methoxymethyl-indan-1-yl]-ethoxy}-diphenylsilane (0.1 g, 0.156 mmol) was dissolved in 4 ml THF, and to this solution was added 1M tetrabutylammonium fluoride solution in THF (312 11, 0.312 mmol). The mixture was stirred under argon at room temperature. Thin layer chromatography in AcOEt and hexane (1:1) was performed. This mixture was extracted with 10 ml of saturated solution of sodium bicarbonate. The organic layer was dried over MgSO₄ and then evaporated. Purification of the residue with flash chromatography using an ethyl acetate/hexane gradient gave 780 mg (95%) of a white powder, AV-176, which is a compound of formula (9) of Scheme 3.

ESIMS, m/z for C₂₃H3006 [M+Na]⁺:425.2

### Example 35

### Methanesulfonic Acid 2-[3-(3,4)-dimethoxyphenyl)-5,6-dimethoxy-2-methoxymethyl-indane-1-yl] ethyl ester

### AV-177

To 10 ml of anhydrous dichloromethane was added AV-176, 2-[3[3,4-dimethoxyphenyl)-5,6-dimethoxy-2-methoxymethyl-indan-1-yl]-ethanol, (0.75 g, 1.86 mmol), methanesulfonyl anhydride (0.487 g, 2.79 mmol), and pyridine (0.22 ml, 2.79 mmol). This mixture was stirred at room temperature under argon overnight. To the mixture was added 50 ml of saturated solution of sodium bicarbonate. The aqueous layer was then extracted with dichloromethane. The combined organic layer was dried over MgSO₄, and evaporated. The residue was purified by flash chromatography using an ethyl acetate/hexane gradient over silica gel to give 570 mg (63%) of a white powder, AV-177, which is compound of formula (10).

1H NMR (300 MHz, DMSO d6) δ 1.8 (m, 1H), 2.2 (m, 1H), 2.6 (m, 1H), 3.03 (s, 3H), 3.29 (s, 3H), 3.40 (m, 2H), 3.53 (t, 1H), 3.74 (s, 3H), 3.80 (s, 3H), 3.88 (s, 3H), 3.94 (d, 1H), 4.37 (m, 2H), 6.38 (s, 1H), 6.73-6.87 (m, 4H). ESIMS, m/z for C₂₄H32O8S [M+Na]⁺:503.2

### Example 36

### 1-Benzyl-4-{2-[3-(3,4-dimethoxyphenyl)-5,6-dimethoxy-2-methoxymethyl-indan-1-yl]-ethyl}-piperazine

### AV-178

AV-177, methanesulfonic acid 2-[3-(3,4)-dimethoxyphenyl)-5,6-dimethoxy-2-methoxymethyl-indane-1-yl] ethyl ester (0.1 g, 0.2 mmol) was added to 4 ml of acetone under argon. To this mixture was added potassium carbonate (77 mg, 0.56 mmol) and 4-benzylpiperazine (0.26 mmol). The resulting mixture was refluxed overnight. The reaction was cooled to room temperature and 5 ml of saturated solution of potassium carbonate was added, and was extracted 3 times with 20 ml of EtOAc. The combined organic layer was dried over MgSO₄ and evaporated. Purification by flash chromatography over silica gel using an ethyl acetate/hexane gradient gave a white powder (70 mg) in 69.5% yield, which is a compound of formula (11).

¹H NMR (300 MHz, DMSO d6) δ 1.48 (m, 1H), 2.01 (m, 1H), 2.50 (m, 8H), 2.67 (m, 2H), 3.28 (s, 3H), 3.34 (m, 2H), 3.52 (s, 3H), 3.55 (m, 2H), 3.71 (s, 3H), 3.79 (s, 3H), 3.87 (s, 6H), 3.93 (d, 1H), 6.38 (s, 1H), 6.61-6.82 (m, 4H), 7.27 (m, 5H). ESIMS, m/z for C₃₄H44N₂O₅ [M+H]+: 503.2.

### Example 37

### 1{-2-[3-(3,4-dimethoxyphenyl)-5,6-dimethoxy-2-methoxymethyl-indan-1-yl]-ethyl}-4-(2-ethoxyphenyl)-piperazine

### AV-194

Prepared analogously to Example 36 from AV-177 and 2-ethoxyphenylpiperazine in a yield of 50% (60 mg), a white powder was obtained.

ESIMS, m/z for C₃₄H44N₂O₅ [M+H]⁺:503.2

### Example 38

### 1-Benzyl-4-{2-[3-(3,4-dimethoxyphenyl)-5,6-dimethoxy-2-methoxymethyl-indan-1-yl]-ethyl}-piperidine

### AV-197

Prepared analogously to Example 36 from AV-177 and 4-benzylpiiperidine in a yield of 55% (32 mg), off-white crystals were obtained.

ESIMS, m/z for C₃₅H₄₅NO₅ [M+H]⁺:560.4

### Example 39

### 1{-2-[3-(3,4-Dimethoxyphenyl)-5,6-dimethoxy-2-methoxymethyl-indan-1-yl]-ethyl}-4-phenylpiperazine

### AV-198

Prepared analogously to Example 36 from AV-177 and 4-phenylpiperazine in a yield of 60% (34 mg), off-white crystals were obtained.

ESIMS,m/z for C₃₃H₄₂N₂O₅ [M+H]⁺:546.7

### Example 40

### 1{-2-[3-(3,4-Dimethoxyphenyl)-5,6-dimethoxy-2-methoxymethyl-indan-1-yl]-ethyl}-4-(4-methoxyphenyl)-piperazine

### AV-199

Prepared analogously to Example 36 from AV-177 and 4-methoxyphenyl-piperazine in a yield of 53% (32 mg), a light tan powder was obtained.

ESIMS,m/z for C₃₄H₄₄N₂O₆ [M+H]⁺:577.4S

### Example 41

### AV-200

### 4-{2-[3-(3,4-Dimethoxyphenyl)-5,6-dimethoxy-2-methoxymethyl-indan-1-yl]-ethyl}-piperazine-1-carboxylic acid benzyl ester.

Prepared analogously to Example 36 from AV-177 and piperazine 1-carboxylic acid benzyl ester in a yield of 46% (29 mg), white crystals were obtained.

ESIMS,m/z for C₃₅H₄₄N₂O₇ [M+H]⁺:503.2

### Example 42

### AV-201

### 1{-2-[3-(3,4-Dimethoxyphenyl)-5,6-dimethoxy-2-methoxymethyl-indan-1-yl]-ethyl}-4-(4-fluorophenyl)-piperazine

Prepared analogously to Example 36 from AV-177 and 2-ethoxyphenylpiperazine in a yield of 41% (24 mg), a white powder obtained. ESIMS,m/z for C₃₅H₄₁N₂FO₅ [M+H]⁺:503.2

### Example 43

### AV-202

### 1-{-2-[3-(3,4-Dimethoxyphenyl)-5,6-dimethoxy-2-methoxymethyl-indan-1-yl]-ethyl}-4-(2-methoxyphenyl)-piperazine

Prepared analogously to Example 36 from AV-177 and 4-(2-methoxyphenyl)-piperazine in a yield of 25% (15 mg), white crystals were obtained. ESIMS,m/z for C₃₄H₄₄N₂O₆ [M+H]⁺:576.7

### Example 44

### AV-203

### 1-[4-(4-{2-[3-3,4-Dimethoxyphenyl)-5,6-dimethoxy-2-methoxymethyl-indan-1-yl]-ethyl}-piperazin-1-yl)-phenyl]-ethanone

Prepared analogously to Example 36 from AV-177 and 4-(1-piperazinylphenyl) ethanone in a yield of 18% (11 mg), white crystals were obtained. ESIMS,m/z for C₃₅H₄₄N₂O₆ [M+H]⁺:588.7

### Example 45

### 1-(2-[3-(3,4-Dimethoxyphenyl)-5,6-dimethoxy-2-methoxymethy-indan-1-yl]-ethyl}-4-(3-phenylallyl)-piperazine

### AV-204

Prepared analogously to Example 36 from AV-177 and 4-(3-phenylallyl)-piperazine in a yield of 41% (25 mg), white crystals were obtained. ESIMS, m/z for C₃₆H₄₆N₂O₅ [M+H]+: 588.7

### Example 46

### 5-benzyloxy-3-(4-benzyloxy-3-methoxy-phenyl)-6-methoxy-1-methoxycarbonyl methyl indane-2 Carboxylic Acid Methyl Ester

A solution of 5-hydroxy-3-(4-hydroxy-3 methoxy-phenyl)-6-methoxy-1-methoxycarbonylmethyl-indane-2carboxylic acid methyl ester (1gr, 2.4 mmol) in 30 ml acetone was prepared. Under argon, potassium carbonate (1 gr, 7.2 mmol), and benzyl bromide (0.86 ml, 7.2 mmol) were added. Then the mixture was stirred at 80° C. overnight. The reaction mixture was cooled, and acetone removed by evaporation. Added 50 ml saturated solution of sodium bicarbonate. The aqueous layer was washed three times with 10 ml ethyl acetate. The combined organic layer was dried over MgSO₄. And evaporated under vacuum. Recrystallization with methanol lead to 1.2 gr of powder.

Y: 84%, ESIMS, m/z for C₃₆H₃₆O₈ [M+Na]+: 619.2

### Example 47

### 5-Benzyloxy-3-(4-benzyloxy-3-methoxy-phenyl)-1-carboxymethyl-6-methoxy-indane-2-carboxylic acid.

A solution of 5-benzyloxy-3-(4-benzyloxy-3-methoxy-phenyl)-6-methoxy-1-methoxycarbonyl methyl indane-2 carboxylic acid methyl ester (0.1 gr, 0.168 mmol) in 1 ml of mixture of solvent THF:H₂O in a ratio of 2:1 was prepared. Under argon, lithium hydroxide (12 mg, 0.49 mmol) was added. The mixture was stirred at 75° C. overnight. Few ml of water was added and aqueous layer was extracted with ethyl acetate. The aqueous layer was acidified, and saturated with sodium chloride, then the saturated aqueous layer was extracted with 10 ml ethyl acetate 3 times, the combined organic layer was dried over MgSO4, filtered and evaporated under vacuum. 69 mg of compound was obtained.

Y: 72%

1H NMR (300 MHz, CDCl₃) δ 4.18(dd, 1H), 2.90(dd, 1H), 3.28 (m, 1H), 3.70 (s, 3H), 3.90

(s, 3H), 3.88-4.2 (m, 1H), 4.6 (d, 1H), 4.9-5.02 (m, 2H), 5.2 (s, 2H), 6.4 (s, 1H), 6.60-6.80 (dd, 4H), 7.2-7.5 (m, 10H).

### Example 48

### 2-{5-benzyloxy-3-(4-benzyloxy-3-methoxy-phenyl)-2-{4-(2-ethoxy-phenyl)-piperazine-1-carbonyl}-6-methoxy-indan-1-yl}-1-(4-(2-ethoxy-phenyl)-piperazin-1-yl]-ethanone

### AV-205

A solution of 5-benzyloxy-3-(4-benzyloxy-3-methoxy-phenyl)-1-carboxymethyl-6-methoxy-indane-2-carboxylic acid (0.05 gr, 0.088 mmol), 1-ethyl-3-(3-dimethylaminopropyl)carbodiimide hydrochloride (0.067 gr, 0.352 mmol), triethylamine (0.049 ml, 0.352 mmol) and dimethylaminopyridine (0.003 gr, 0.026 mmol) in 3 ml THF was prepared. The resulting mixture was stirred under argon at room temperature for 3 hours. 1-(2-ethoxyphenyl)piperazine hydrochloride (0.085 gr, 0.352 mmol) was added to the mixture. The resulting mixture was stirred overnight at room temperature. The white precipitate was filtered. The crude filtered solution was washed with a saturated solution of sodium bicarbonate. The aqueous layer was extracted with dichloromethane and the combined organic layers were dried over MgSO₄ and evaporated. Flash chromatography of the residue over silica gel using an ethyl acetate/hexane gradient gave 7 mg of a clear oil.

Clear oil, Y: 8% ESIMS, m/z for C₅₈H₆₄N₄O₈ [M]+: 945.166

AV-206: 2-[2-[4-(2-ethoxy-phenyl)-piperazine-1-carbonyl]-5-hydroxy-3-(4-hydroxy-3-methoxy-phenyl)-6-methoxy-inda-1-yl]-1-[4(2-ethoxy-phenyl)-piperazin-1-yl]-ethanone

### Example 49

1-Carboxymethyl-5-hydroxy-3-(4-hydroxy-3-methoxy-phenyl)-6-methoxy-indan-2-carboxylicacid

A solution of 5-hydroxy-3-(4-hydroxy-3-methoxy-phenyl)-6-methoxy-1methoxycarbonyl methyl indane-2 carboxylic acid methyl ester (0.2 gr, 0.480 mmol) in 1 ml of mixture of solvent THF:H₂O in a ratio of 2:1 was prepared. Under argon, lithium hydroxide (51 mg, 2.11 mmol) was added. The mixture was stirred at 75° C. overnight. Few ml of water was added and aqueous layer was extracted with ethyl acetate. The aqueous layer was acidified, and saturated with sodium chloride, then the saturated aqueous layer was extracted with 10 ml ethyl acetate 3 times, the combined organic layer was dried over MgSO₄, filtered and evaporated under vacuum. 118 mg of compound was obtained.

Y: 63% ESIMS, m/z for C₂₀H₂₀O₈ [M+Na]+: 411

### Example 50

2-[2-[4-(2-ethoxy-phenyl)-piperazine-1-carbonyl]-5-hydroxy-3-(4-hydroxy-3-methoxy-phenyl)-6-methoxy-inda-1-yl]-1-[4(2-ethoxy-phenyl)-piperazin-1-yl]-ethanone

### AV-206

A solution of 1-carboxymethyl-5-hydroxy-3-(4-hydroxy-3-methoxy-phenyl)-6-methoxy-indan-2-carboxylicacid (0.118 gr, 0.304 mmol), 1-ethyl-3-(3-dimethylaminopropyl)carbodiimide hydrochloride (0.234 gr, 1.22 mmol), triethylamine

(0.170 ml, 1.22 mmol) and dimethylaminopyridine (0.011 gr, 0.091 mmol) in 3 ml THF was prepared. The resulting mixture was stirred under argon at room temperature for 3 hours. 1-(2-ethoxyphenyl)piperazine hydrochloride (0.296 gr, 1.22 mmol) was added to the mixture. The resulting mixture was stirred overnight at room temperature. The white precipitate was filtered. The crude filtered solution was washed with a saturated solution of sodium bicarbonate. The aqueous layer was extracted with dichloromethane and the combined organic layers were dried over MgSO₄ and evaporated. Flash chromatography of the residue over silica gel using an ethyl acetate/hexane gradient gave 3 mg of a white powder.

Y; 1% ESIMS, m/z for C₄₄H5208N4 [M+H] 765

1H NMR (300 MHz, CDCl₃) δ 1.28(t, 3H), 2.20(t, 3H), 2.48 (dd, 1H), 2.88 (dd, 1H),3.0-3.45 (m, 16H), 3.60 (m, 1H), 3.81 (s, 3H), 3.9 (s, 3H), 4.0-4.2 (m, 3H), 4.82 (d, 1H), 6.42 (s, 1H), 6.7-7.0 (m, 7H), 7.18-7.3 (m, 5H)

AV-207: 2-{5-ethoxy-3-(4-ethoxy-3-methoxy-phenyl)-2-[4-(2-ethoxy-phenyl)-piperazine-1-carbonyl]-6-methoxy-indan-1-yl}-1-[4-(2-ethoxy-phenyl)-piperazin-1-yl]-ethanone

### Example 51

5-ethoxy-3-(4-ethoxy-3-methoxy-phenyl)-6-methoxy-1-methoxycarbonylmethyl-indan-2-carboxylic Acid Methyl Ester

A solution of 5-hydroxy-3-(4-hydroxy-3 methoxy-phenyl)-6-methoxy-1-methoxycarbonylmethyl-indane-2carboxylic acid methyl ester (1gr, 2.4 mmol) in 30 ml acetone was prepared. Under argon, potassium carbonate (1 gr, 7.2 mmol), and iodo-ethane (0.576 ml, 7.2 mmol) were added. Then the mixture was stirred at 80° C. overnight. The reaction mixture was cooled, and acetone removed by evaporation. Added 50 ml saturated solution of sodium bicarbonate. The aqueous layer was washed three times with 10 ml ethyl acetate. The combined organic layer was dried over Mg SO₄. And evaporated under vacuum. Recrystallization with methanol lead to 1.2 gr of powder.

Y: 25%, ESIMS, m/z for C₂₆H3208 [M+Na]⁺: 495.1

### Example 52

### 1-Carboxymethyl-5-ethoxy-3-(4-ethoxy-3-methoxy-phenyl)-6-methoxy-indan-2-carboxylic Acid

A solution of 5-ethoxy-3-(4-ethoxy-3-methoxy-phenyl)-6-methoxy-1-methoxycarbonylmethyl-indan-2-carboxylic acid methyl ester (0.325 gr, 0.688 mmol) in 3 ml of mixture of solvent THF:H₂O in a ratio of 2:1 was prepared. Under argon, lithium hydroxide (36 mg, 1.51 mmol) was added. The mixture was stirred at 75° C. overnight. Few ml of water was adde d and aqueous layer was extracted with ethyl acetate. The aqueous layer was acidified, and saturated with sodium chloride, then the saturated aqueous layer was extracted with ethyl acetate 3 times, the combined organic layer was dried over MgSO4, filtered 10 ml and evaporated under vacuum. 69 mg of compound was obtained.

Y: 90% ESIMS, m/z for C₂₄H₂₈O₈ [M+Na]⁺: 467.1

### Example 53

### 2-{5-ethoxy-3-(4-ethoxy-3-methoxy-phenyl)-2-[4-(2-ethoxy-phenyl)-piperazine-1-carbonyl]-6-methoxy-indan-1-yl}-1-[4-(2-ethoxy-phenyl)-piperazin-1-yl]-ethanone

### AV-207

A solution of 1-carboxymethyl-5-ethoxy-3-(4-ethoxy-3-methoxy-phenyl)-6-methoxy-indan-2-carboxylic acid (0.100 gr, 0.225 mmol), 1-ethyl-3-(3-dimethylaminopropyl)carbodiimide hydrochloride (0.173 gr, 0.9 mmol), triethylamine

(0.125 ml, 0.9 mmol) and dimethylaminopyridine (0.008 gr, 0.068 mmol) in 3 ml THF was prepared. The resulting mixture was stirred under argon at room temperature for 3 hours. 1-(2-ethoxyphenyl)piperazine hydrochloride (0.221 gr, 0.9 mmol) was added to the mixture. The resulting mixture was stirred overnight at room temperature. The white precipitate was filtered. The crude filtered solution was washed with a saturated solution of sodium bicarbonate. The aqueous layer was extracted with dichloromethane and the combined organic layers were dried over MgSO₄ and evaporated. Flash chromatography of the residue over silica gel using an ethyl acetate /hexane gradient gave 19 mg of a white powder.

Y; 10% ESIMS, m/z for C₄₈H₆₀O₈N₄ [M+H] 821.5

1H NMR (300 MHz, CDCl₃) δ 1.30(t, 3H), 1.32-1.50(m, 9H), 2.52 (dd, 1H), 2.70

(dd, 1H),2.78-2.903 (m, 1H), 2.92-3.61 (m, 8H), 3.09-3.80 (m, 1H), 3.32-3.42 (m, 1H), >03.5-3.78 (m, 8H), 3.8(s, 3H), 3.82 (s, 3H), 3.89-3.98 (m, 2H), 4.2-4.18 (m, 6H), 5.0 (d, 1H), 6.4 (s, 1H), 6.72-7.2 (m, 12H)

AV-208: 2-{5-butoxy-3-(4-butoxy-3-methoxy-phenyl)-2-[4-(2-ethoxy-phenyl)-piperazine-1-carbonyl]-6-methoxy-indan-1-yl}-1-[4-(2-ethoxy-phenyl)-piperazin-1-yl]-ethanone

### Example 54

### 5-Butoxy-3-(4-butoxy-3-methoxy-phenyl)-6-methoxy-1-methoxycarbonylmethyl-indan-2-carboxylic Acid Methyl Ester

A solution of 5-hydroxy-3-(4-hydroxy-3 methoxy-phenyl)-6-methoxy-1-methoxycarbonylmethyl-indane-2carboxylic acid methyl ester (1 gr, 2.4 mmol) in 30 ml acetone was prepared. Under argon, potassium carbonate (1 gr, 7.2 mmol), and 1-iodobutane (0.819 ml, 7.2 mmol) were added. Then the mixture was stirred at 80° C. overnight. The reaction mixture was cooled, and acetone removed by evaporation. Added 50 ml saturated solution of sodium bicarbonate. The aqueous layer was washed three times with 10 ml ethyl acetate. The combined organic layer was dried over Mg SO₄. And evaporated under vacuum. Recrystallization with methanol lead to 459 mg of powder.

Y; 36% ESIMS, m/z for C₃₀H4008 [M+Na]⁺: 551.2

### Example 55

### 5-Butoxy-3-(4-butoxy-3-methoxy-phenyl)-1-carboxymethyl-6-methoxy-indan-2-carboxylic Acid

A solution of 5-butoxy-3-(4-butoxy-3-methoxy-phenyl)-6-methoxy-1-methoxycarbonylmethyl-indan-2-carboxylic acid methyl ester (0.455 gr, 0.861 mmol) in 5 ml of mixture of solvent THF: H₂O in a ratio of 2:1 was prepared. Under argon, lithium hydroxide (45 mg, 1.89 mmol) was added. The mixture was stirred at 750 C overnight. Few ml of water was added and aqueous layer was extracted with ethyl acetate. The aqueous layer was acidified, and saturated with sodium chloride, then the saturated aqueous layer was extracted with 10 ml ethyl acetate 3 times, the combined organic layer was dried over MgSO₄, filtered and evaporated under vacuum. 353 mg of compound was obtained.

Y: 81% ESIMS C₂₈H₃₆O_{8 [2}M+Na]⁺: 1024.4

### Example 56

2-{5-butoxy-3-(4-butoxy-3-methoxy-phenyl)-2-[4-(2-ethoxy-phenyl)-piperazine-1-carbonyl]-6-methoxy-indan-1-yl}-1-[4-(2-ethoxy-phenyl)-piperazin-1-yl]-ethanone

### AV-208

A solution of 5-butoxy-3-(4-butoxy-3-methoxy-phenyl)-1-carboxymethyl-6-methoxy-indan-2-carboxylic acid (0.100 gr, 0.194 mmol), 1-ethyl-3-(3-dimethylaminopropyl)carbodiimide hydrochloride (0.149 gr, 0.777 mmol), triethylamine (0.108 ml, 0.777 mmol) and dimethylaminopyridine (0.007 gr, 0.058 mmol) in 3 ml THF was prepared. The resulting mixture was stirred under argon at room temperature for 3 hours. 1-(2-ethoxyphenyl)piperazine hydrochloride (0.191 gr, 0.777 mmol) was added to the mixture. The resulting mixture was stirred overnight at room temperature. The white precipitate was filtered. The crude filtered solution was washed with a saturated solution of sodium bicarbonate. The aqueous layer was extracted with dichloromethane and the combined organic layers were dried over MgSO₄ and evaporated. Flash chromatography of the residue over silica gel using an ethyl acetate /hexane gradient gave 2 isomers.

Isomer-1: 12 mg 7% ESIMS C₅₂H₆₈O₈N₄ [M+H]: 877

1H NMR (300 MHz, CDCl₃) δ 0.84-1.0 (m, 5H), 1.30-1.64(m, 9H), 1.68-1.86 (m, 4H), 2.70 (dd, 1H),2.70 (dd, 1H), 2.76-2.98 (m, 2H), 2.72-3.04 (m, 4H), 3.09-3.18 (m, 1H), 3.32-3.42 (m, 1H), 3.5-3.9(m, 16H), 3.88-4.18 (m, 8H), 5 (d, 1H) 6.4 (s, 1H), 6.72-7.2

(m, 12H)

Isomer-2: 12 mg 7% ESIMS C₅₂H₆₈O₈N₄ [M+H]: 877

### Example 58

### 5-Decyloxy-3-(4-decyloxy-3-methoxy-phenyl)-6-methoxy-1-methoxycarbonylmethyl-indan-2-carboxylic Acid Methyl Ester

A solution of 5-hydroxy-3-(4-hydroxy-3 methoxy-phenyl)-6-methoxy-1-methoxycarbonylmethyl-indane-2carboxylic acid methyl ester (1 gr, 2.4 mmol) in 30 ml acetone was prepared. Under argon, potassium carbonate (2 gr, 14.4 mmol), and 1-iodecane (3.1 ml, 14.4 mmol) were added. Then the mixture was stirred at 80° C. overnight. The reaction mixture was cooled, and acetone removed by evaporation. Added 50 ml saturated solution of sodium bicarbonate. The aqueous layer was washed three times with 10 ml ethyl acetate. The combined organic layer was dried over MgSO₄. And evaporated under vacuum. Flash chromatography over silica gel using ethyl acetate 5%/hexane gave 100 mg of pure compound and more impure fractions.

Y: 14%

### Example 59

### 1-carboxymethyl-5-decyloxy-3-(4-decyloxy-3-methoxy-phenyl)-6-methoxy-indan-2-carboxylic acid.

A solution of 5-Decyloxy-3-(4-decyloxy-3-methoxy-phenyl)-6-methoxy-1-methoxycarbonylmethyl-indan-2-carboxylic acid methyl ester (0.100 gr, 0.143 mmol) in 5 ml of mixture of solvent THF:H₂O in a ratio of 2:1 was prepared. Under argon, lithium hydroxide (8 mg, 0.315 mmol) was added. The mixture was stirred at 75° C. few hours. Few ml of water was added and aqueous layer was extracted with ethyl acetate. The aqueous layer was acidified, and saturated with sodium chloride, then the saturated aqueous layer was extracted with ethyl acetate 10 ml 3 times, the combined organic layer was dried over MgSO₄, filtered and evaporated under vacuum. 83 mg of compound was obtained.

Y: 86%

### Example 60

### 2-{5-Decyloxy-3-(4-decyloxy-3-methoxy-phenyl)-2-[4-(2-ethoxy-phenyl)-piperazine-1-carbonyl]-6-methoxy-indan-1-yl}-1-[4-(2-ethoxy-phenyl)-piperazin-1-yl]-ethanone

### AV-209

A solution of 1-carboxymethyl-5-decyloxy-3-(4-decyloxy-3-methoxy-phenyl)-6-methoxy-indan-2-carboxylic acid. (0.083 gr, 0.124 mmol), 1-ethyl-3-(3-dimethylaminopropyl)carbodiimide hydrochloride (0.095 gr, 0.496 mmol), triethylamine (0.069 ml, 0.49 mmol) and dimethylaminopyridine (0.004 gr, 0.037 mmol) in 3 ml THF was prepared. The resulting mixture was stirred under argon at room temperature for 3 hours. 1-(2-ethoxyphenyl)piperazine hydrochloride (0.122 gr, 0.496 mmol) was added to the mixture. The resulting mixture was stirred overnight at room temperature. The white precipitate was filtered. The crude filtered solution was washed with a saturated solution of sodium bicarbonate. The aqueous layer was extracted with dichloromethane and the combined organic layers were dried over MgSO₄ and evaporated. Flash chromatography of the residue over silica gel using an ethyl acetate /hexane gradient gave 7 mg of a white powder.

Y; 5% ESIMS, m/z for C₆₄H₉₂O₈N₄ [M+H]: 1045.45

1H NMR (300 MHz, CDCl₃) δ 0.84-1.0 (m, 7H), 1.10-1.45(m, 29H), 1.5-1.88 (m, 6H), 2.70 (dd, 1H),2.70 (dd, 1H), 2.76-2.98 (m, 2H), 2.72-3.04 (m, 4H), 3.09-3.18 (m, 1H), 3.32-3.42 (m, 1H), 3.5-3.9(m, 16H), 3.88-4.18 (m, 8H), 5 (d, 1H) 6.4 (s, 1H), 6.72-7.2

(m, 12H)

### Example 61

2-[3-(3,4-Dimethoxy-phenyl)-2-hydroxymethyl-5,6-dimethoxy-indan-1-yl]-ethanol

A solution of 1-(3,4-Dimethoxy-phenyl)-5,6-dimethoxy-3-methoxycarbonylmethyl-indan-2-carboxylic acid methyl ester (13.3 g, 29.9 mmol), in 200 ml THF was prepared. Lithium Aluminum hydride (100 ml, 89.8 mmol) was added slowly to the mixture. The resulting mixture was stirred 3 hours at 70° C. under argon. The mixture was cooled to 0° C. and ethyl acetate drop by drop was added until no more bubble is produce in the reaction mixture. 100 ml of water was added and the mixture stirred 1 hour at room temperature. The aqueous layer was extracted with ethyl acetate few times and the combined organic layers were dried over MgSO₄ and evaporated. Crystallization with benzene lead to 9.3 g white powder.

Y: 80%

### Example 62

### Methanesulfonic acid 2-[3-(3,4-dimethoxy-phenyl)-2-methansufonyloxymethyl-5,6-dimethoxy-indan-1-yl]-ethyl Ester

A solution of 2-[3-(3,4-Dimethoxy-phenyl)-2-hydroxymethyl-5,6-dimethoxy-indan-1-yl]-ethanol. (6 g, 15.4 mmol) in 100 ml anhydrous dichloromethane was prepared. Methanesulfonic anhydride (8.1 g, 46.3 mmol) and pyridine (3.74 ml, 46.3 mmol) were added to the mixture. The resulting mixture was stirred overnight at room temperature. The white precipitate was filtered. The crude filtered solution was washed with a saturated solution of sodium bicarbonate. The aqueous layer was extracted with dichloromethane and the combined organic layers were dried over MgSO₄ and evaporated. Flash chromatography of the residue over silica gel using an ethyl acetate gave 7.2 g of white powder.

Y; 86% ESIMS, m/z for C₂₄H₃₂O₁₀S₂ [M+Na]: 567.14

### Example 63

### 1-(2-Azido-ethyl)-2-azidomethyl-3-(3,4-dimethoxy-phenyl)-,6-dimethoxy-indan

A solution of methanesulfonic acid 2-[3-(3,4-dimethoxy-phenyl)-2-methansufonyloxymethyl-5,6-dimethoxy-indan-1-yl]-ethyl ester (2 g, 3.67 mmol) in 10 ml anhydrous dimethylformamide was prepared. Sodium azide (0.71 g, 11.02 mmol) was added. The resulting mixture was stirred at 70° C. 3 hours under argon. The crude mixture was washed with a saturated solution of sodium bicarbonate. The aqueous layer was extracted with ethyl acetate and the combined organic layers were dried over MgSO4 and evaporated. Flash chromatography of the residue over silica gel using an ethyl acetate and hexane 10% gave 1.7 g of white powder.

Y; 100% ESIMS, m/z for C₂₂H₂₆O₄N₆[M+Na]⁺: 461.2

1H NMR (300 MHz, CDCl3) δ 1.78-1.88 (m, 2H), 1.39-1.45(m, 2H), 2.78-3.00 (m, 4H), 3.25 (m, 4H), (dd, 1H),3.7 (s, 3H), 3.8 (s, 3H), 3.9 (s, 6H), 6.4 (s, 1H), 6.6-7.85 (m, 4H)

### Example 64 2-[2-aminomethyl-3-(3,4-dimethoxy-phenyl)-5,6-dimethoxy-indan-1-yl]-ethylamine

A solution of 1-(2-Azido-ethyl)-2-azidomethyl-3-(3,4-dimethoxy-phenyl)-5,6-dimethoxy-indan (1 g, 2.28 mmol) in 40 ml anhydrous methanol was prepared. Palladium on carbon 10% (150 mg) was added. The resulting mixture was stirred at room temperature overnight under hydrogen. The paladium was filtered. The crude mixture was washed with methanol 100 ml. Methanol was evaporated, 5 ml of HCl 1 N was added to the crude material and the acidic aqueous layer was extracted 3 times with 20 ml dichloromethane. The aqueous layer was then cooled at 0° C. and sodium hydroxide 1 N was added until the pH is basic, extracted with dichloromethane and the combined organic layers were dried over MgSO4 and evaporated, gave 300 mg of white compound.

Y: 34%

### Example 65

### N-{2-[(2-N-methyl)-benzenesulfonamide-3-(3,4-dimethoxy-phenyl)-5,6-dimethoxy-inda-1-yl}-ethyl}-benzensulfonamide

### AV-223

A solution of 2-[2-aminomethyl-3-(3,4-dimethoxy-phenyl)-5,6-dimethoxy-indan-1-yl]-ethylamine (0.03 g, 0.07 mmol), in 3 ml anhydrous dichloromethane was prepared. To this solution was added morpholinomethyl polystyrene resin (0.2 g. reference Novobiochem 3.50 mmol/g). Benzensulfonyl chloride (0.049 g, 0.28 mmol) was added to the suspension. The mixture was shacked at room temperature overnight. The polystyrene resin was then filtered and washed with dichloromethane. To the dichloromethane solution the polyamine resin the tris(2-aminoethylamine), (200 mg from Novabiochem). The suspension was shaken at room temperature overnight, then the mixture was filtrated. The dichloromethane was evaporated under vacuum gave 27 mg of white powder.

Y; 29% ESIMS, m/z for C₃₄H₃₈O₈N₂S₂ [M+Na]⁺: 689

### Example 66

### 10-Benzyl-3-{2-[2-(3-benzyl-ureidomethyl)-3-(3,4-dimethoxy-phenyl)-5,6-dimethoxy-inda-1-yl]-ethyl}-urea

### AV-234

A solution of 2-[2-aminomethyl-3-(3,4-dimethoxy-phenyl)-5,6-dimethoxy-indan-1-yl]-ethylamine (0.02g, 0.05 mmol) in 0.5 ml anhydrous dichloromethane was prepared. Benzyl isocyanate (0.024 ml, 0.2 ml) was added. Then the mixture was stirred room temperature for an hour. To this mixture was added a suspension of polyamine resin the tris(2-aminomethylamine), (150 mg from Novabiochem) in 2 ml of dichloromethane. After 2 hour filter the resin, and dichloromethane was evaporated under vacuum gave 18 mg of white compound.

White powder Y: 27% ESIMS, m/z for C₃₈H₄₄O₆N₄ [M+H]⁺: 653.4

### Example 67

### 5-Methoxy-3-methoxycarbonylmethyl-1-(3-methoxy-4-propoxy-phenyl)-6-propoxy-indan-2-carboxylic Acid Methyl Ester

A solution of 5-hydroxy-3-(4-hydroxy-3 methoxy-phenyl)-6-methoxy-1-methoxycarbonylmethyl-indane-2carboxylic acid methyl ester (0.5 g, 1.2 mmol) in 15 ml acetone was prepared. Under argon, potassium carbonate (0.664 gr, 4.80 mmol), and 1-iodopropane (0.46 ml, 4.80 mmol) were added. Then the mixture was stirred at 80° C. overnight. The reaction mixture was cooled, and acetone removed by evaporation. Added 50 ml saturated solution of sodium bicarbonate. The aqueous layer was washed three times with 10 ml ethyl acetate. The combined organic layer was dried over MgSO4 and evaporated under vacuum. Chromatotor purification over silica gel using a gradient of ethyl acetate/hexane gave 0.459 g of brown crystals.

Y: 76%.

### Example 68

### 1-Carboxymethyl-6-methoxy-3-(3-methoxy-4-propoxy-phenyl)-5-propoxy-indan-2-carboxylic Acid

A solution of 5-Methoxy-3-methoxycarbonylmethyl-1-(3-methoxy-4-propoxy-phenyl)-6-propoxy-indan-2-carboxylic acid methyl ester (0.459 g, 0.917 mmol) in 5 ml of mixture of solvent THF:H₂O in a ratio of 2:1 was prepared. Under argon, lithium hydroxide (48 mg, 2.02 mmol) was added. The mixture was stirred at 75° C. few hours. Few ml of water was added and aqueous layer was extracted with ethyl acetate. The aqueous layer was acidified, and saturated with sodium chloride, then the saturated aqueous layer was extracted with 10 ml ethyl acetate 3 times, the combined organic layer was dried over MgSO₄, filtreted and evaporated under vacuum. 0.4 g of light brown powder was obtained.

Y: 93%

### Example 69

### 2-[2-4-(2-ethoxy-phenyl)-piperazine-1-carbonyl]-6-methoxy-3-(3-methoxy-4-propoxy-phenyl)-5-propoxy-inda-1-yl]-1-[4-2(ethoxy-phenyl)-piperazin-1-yl]-ethanone

### AV-239

A solution of 1-Carboxymethyl-6-methoxy-3-(3-methoxy-4-propoxy-phenyl)-5-propoxy-indan-2-carboxylic acid. (0.20 g, 0.423 mmol), 1-ethyl-3-(3-dimethylaminopropyl)carbodiimide hydrochloride (0.324 g, 1.69 mmol), triethylamine (0.236 ml, 1.69 mmol) and dimethylaminopyridine (0.015 g, 0.127 mmol) in 10 ml THF was prepared. The resulting mixture was stirred under argon at room temperature for 3 hours. 1-(2-ethoxyphenyl)piperazine hydrochloride (0.415 g, 1.69 mmol) was added to the mixture. The resulting mixture was stirred overnight at room temperature. The white precipitate was filtered. The crude filtered solution was washed with a saturated solution of sodium bicarbonate. The aqueous layer was extracted with dichloromethane and the combined organic layers were dried over MgSO₄ and evaporated. Flash chromatography over silica gel with a gradient ethyl-acetate/hexane isolated the isomer 1. Chromatoturn on mixture of isomer 1 and 2 with elution ethyl acetate/hexane 1:1 gave separation of isomer 1 and 2. 69 mg of off white crystals was obtained as isomer 1, and 8mg of clear oil as isomer 2.

Isomer 1: Y: 19% ESIMS, m/z for C₅₀H₆₄O₈N₄ [M+H]: 849.5

1H NMR (300 MHz, CDCl₃) δ 0.9-1.02 (m, 3H), 1.39-1.50(m, 3H), 1.71-1.92 (m, 3H), 2.70 (dd, 1H),2.75 (dd, 1H), 2.76-2.98 (m, 2H), 2.72-3.04 (m, 4H), 3.09-3.18 (m, 1H), 3.32-3.42 (m, 1H), 3.5-3.9(m, 16H), 3.88-4.18 (m, 8H), 5 (d, 1H) 6.4 (s, 1H), 6.72-7.02 (m, 12H)

Isomer 2: Y: 2% ESIMS, m/z for C₅₀H₆₄O₈N₄ [M+H]: 849.5

1H NMR (300 MHz, CDCl₃) δ 0.9-1.02 (m, 3H), 1.39-1.50(m, 3H), 1.71-1.92 (m, 3H), 2.70 (dd, 1H),2.75 (dd, 1H), 2.76-2.98 (m, 2H), 2.72-3.04 (m, 4H), 3.09-3.18 (m, 1H), 3.32-3.42 (m, 1H), 3.5-3.9(m, 16H), 3.88-4.18 (m, 8H), 4.38 (d, 1H) 6.4 (s, 1H), 6.72-7.02 (m, 12H)

### Example 70

### 1-(3,4-Dimethoxy-phenyl)-3-(2-[4-(2-ethoxy-phenyl)-piperazin-1-yl]-2-oxo-ethyl}-5,6-dimethoxy-indan-2-carboxylic acid

### AV-250

A solution of 1-carboxymethyl-3-(3,4-dimethoxy-phenyl)-5,6-dimethoxy-indan-2-carboxylic acid. (1 g, 2.40 mmol), 1-ethyl-3-(3-dimethylaminopropyl)carbodiimide hydrochloride (0525 g, 2.88 mmol), triethylamine (0.401 ml, 2.88 mmol) and dimethylaminopyridine (0.059 g, 0.480 mmol) in 40 ml THF was prepared. The resulting mixture was stirred under argon at room temperature for 3 hours. 1-(2-ethoxyphenyl)piperazine hydrochloride (0.699 g, 2.88 mmol) was added to the mixture. The resulting mixture was stirred overnight at room temperature. Added sodium hydroxide 1 N, extracted the aqueous layer with ethyl acetate. The aqueous layer was then acidified with acetic acid and extracted with ethyl acetate. The combined organic layer of acidic extraction were dried over MgSO₄, and evaporated under vacuum. The mixture was purified using chromatoturn elution with ethyl acetate and methanol 10%. The impure fractions were purified through reverse phase HPLC using a gradient of 25-45% acetonitril gave 200 mg of white compound.

Y: 13% ESIMS, m/z for C₃₄H₄₀O₈N₂[M+H]: 605.3

### Example 71

2-[2-(4-Benzyl-piperidine-1-carbonyl)-5-hydroxy-3-(4-hydroxy-3-methoxy-phenyl)-6-methoxy-indan-1-yl-1-(4-benzyl-piperidin-1-yl)-ethanone

### AV-251

Prepared analogously to example 50 from 1-phenylpiperidine in a yield 6.5% (25 mg), a white powder isomer 1 ,and 5% (20 mg) of an oil isomer 2.

Isomer 1: ESIMS, m/z for C₄₄H₅₀O₆N₂ [M+H]: 704.4

Isomer 2: ESIMS, m/z for C₄₄H₅₀O₆N₂ [M+H]: 704.4

### Example 72

2-[2-[4-(2-Ethoxy-phenyl)-piperazine-1-carbonyl]-6-methoxy-3-(3-methoxy-4-pentyloxy-phenyl)-5-pentyloxy-indan-1-yl]-1-[4-(2-ethoxy-phenyl)-piperazin-1-yl]ethanone

### AV-252

A solution of 2-[2-[4-(2-ethoxy-phenyl)-piperazine-1-carbonyl]-5-hydroxy-3-(4-hydroxy-3-methoxy-phenyl)-6-methoxy-inda-1-yl]-1-[4(2-ethoxy-phenyl)-piperazin-1-yl]-ethanone (0.036 g, 0.04 mmol) in 15 ml acetone was prepared. Under argon, potassium carbonate (0.032 g, 0.235 mmol), and 1-iodopentate (0.031 ml, 0.235 mmol) were added. Then the mixture was stirred at 80° C. overnight. The reaction mixture was cooled, and acetone removed by evaporation. Added 50 ml saturated solution of sodium bicarbonate. The aqueous layer was washed three times with 10 ml ethyl acetate. The combined organic layer was dried over MgSO₄. And evaporated under vacuum. Flash chromatography purification over silica gel using a gradient of ethyl acetate/hexane gave 4 mg of oil.

Y: 10% ESIMS, m/z for C₅₄H₇₂O₈N₄ [M+H]: 905.185

### Example 73

### 1-Carboxymethyl-3-(3,4-diethoxy-phenyl)-5,6-diethoxy-indan-2-carboxylic Acid

A solution of 3-(3,4-Diethoxy-phenyl)-acrylic acid (0.064 g, 0271 mmol) in 2 ml of TFA was stirred at room temperature 5 days. The TFA was evaporated under vacuum. Added 10 ml saturated solution of sodium bicarbonate, and extracted with ethyl acetate. The aqueous layer was acidified with 1 normal HCl. The aqueous acidic layer was extracted with ethyl acetate. The combined organic layer was dried over MgSO4 and evaporated under vacuum. Flash chromatography purification over silica gel using a gradient ethyl acetate/hexane gave 0.036 mg of white powder.

Y: 56% ESIMS, m/z for C₂₆H₃₂O_{8 [2}M+Na]⁺:495.2

1H NMR (300 MHz, CDCl₃) δ 0.9-0.99 (m, 6H), 1.20-1.50 (m, 12H), 1.60-1.92 (m, 6H), 2.50 (dd, 1H),2.75 (dd, 1H), 2.78-2.90 (m, 2H), 2.95-3.10 (m, 4H), 3.1-3.2 (m, 1H), 3.32-3.9 (m, 15H), 3.9-4.19(m, 6H), 5 (d, 1H) 6.42 (s, 1H), 6.72-7.02 (m, 12H)

### Example 74

### 2-{3-93,4-diethoxy-phenyl)-5,6-diethoxy-2-[4-(2-ethoxy-phenyl)-piperazine-1-carbonyl]-indan-1-yl}-1[4-2-ethoxy-phenyl)-piperazine-1-yl]-ethanone

### AV-253

A solution of 1-Carboxymethyl-3-(3,4-diethoxy-phenyl)-5,6-diethoxy-indan-2-carboxylic acid. (0.03 g, 0.063 mmol), 1-ethyl-3-(3-dimethylaminopropyl)carbodiimide hydrochloride (0.048 g, 0.252 mmol), triethylamine (0.035 ml, 0.252 mmol) and dimethylaminopyridine (0.002 g, 0.019 mmol) in 3 ml THF was prepared. The resulting mixture was stirred under argon at room temperature for 3 hours. 1-(2-ethoxyphenyl)piperazine hydrochloride (0.061 g, 0.252 mmol) was added to the mixture. The resulting mixture was stirred overnight at room temperature. The white precipitate was filtered. The crude filtered solution was washed with a saturated solution of sodium bicarbonate. The aqueous layer was extracted with dichloromethane and the combined organic layers were dried over MgSO₄ and evaporated. Flash chromatography over silica gel with a gradient ethyl-acetate/hexane isolated the isomer 1. Chromatoturn on mixture of isomer 1 and 2 with elution ethyl acetate/hexane 1:1 gave separation of isomer 1 and 2.17.5 mg of off yellow solid was obtained as isomer 1, and 1.7 mg of clear oil as isomer 2.

Isomer 1: Y: 19% ESIMS, m/z for C₅₀H640₈N₄ [M+H]: 849.5

1H NMR (300 MHz, CDCl₃) δ 1.3-1.5 (m, 18H) 2.50 (dd, 1H),2.75 (dd, 1H), 2.78-2.90 (m, 2H), 2.95-3.10 (m, 4H), 3.1-3.2 (m, 1H), 3.4-4.2 (m, 20H), 5 (d, 1H) 6.42 (s, 1H), 6.72-7.02 (m, 12H)

Isomer 2: Y: 2% ESIMS, m/z for C₅₀H₆₄O₈N₄ [M+H]: 849.5

### AV-254: 2-{3-(3,4-Dipopoxy-phenyl)-2-[4-(2-ethoxy-phenyl)-piperazine-1-carbonyl]-5,6-dipropoxy-indan-1-yl}-1-[4-(2-ethoxy-phenyl)-piperazin-1-yl]-ethanone

### Example 75

1-Carboxymethyl-3-(3,4-dipropoxy-phenyl)-5,6-dipropoxy-indan-2-carboxylic acid Prepared analogously to example 73 from 3-(3,4-Dipropoxy-phenyl)-acrylic acid in a yield 40% (0.4 g) a white powder.

Y: 40% ESIMS, m/z for C₃₀H₄₀O₈ [M+Na]+: 551.4

### Example 76

### 2-{3-(3,4-Dipopoxy-phenyl)-2-[4-(2-ethoxy-phenyl)-piperazine-1-carbonyl]-5,6-dipropoxy-indan-1-yl}-1-[4-(2-ethoxy-phenyl)-piperazin-1-yl]-ethanone

### AV-254

Prepared analogously to example 74 from 1-Carboxymethyl-3-(3,4-dipropoxy-phenyl)-5,6-dipropoxy-indan-2-carboxylic acid in a yield 5% (8 mg g) a clear solid.

1H NMR (300 MHz, CDCl₃) δ 0.9-1.02 (m, 11H), 1.40-1.42 (m, 5H), 1.60-1.92 (m, 10H), 2.50 (dd, 1H),2.75 (dd, 1H), 2.76-2.98 (m, 2H), 2.72-3.04 (m, 4H), 3.09-3.18 (m, 1H), 3.32-3.42 (m, 1H), 3.54.1(m, 22H), 5 (d, 1H) 6.42 (s, 1H), 6.72-7.02 (m, 12H)

ESIMS, m/z for C₅₄H₇₂O₈N₄ [M+H]⁺: 905.7

### Example 77

### 5,6-Dibutoxy-1-carboxymethyl-3-(3,4-dibutoxy-phenyl)-indan-2-carboxylic acid

Prepared analogously to example 73 from 3-(3,4-Dibutoxy-phenyl)-acrylic acid in a yield 46% (0.618 g) a tan solid.

ESIMS, m/z for C₃₄H₄₈O₈ [M+Na]⁺: 607.3

### Example 78

### 2-{3-(3,4-Dibutoxy-phenyl)-2-[4-(2-ethoxy-phenyl)-piperazine-1-carbonyl]-5,6-dibutoxy-indan-1-yl}-1-[4-(2-ethoxy-phenyl)-piperazin-1-yl]-ethanone

### AV-255

Prepared analogously to example 74 from 5,6-dibutoxy-1-carboxymethyl-3-(3,4-dibutoxy-phenyl)-indan-2-carboxylic acid in a yield 5% (8 mg) a clear solid.

¹H NMR (300 MHz, CDCl3) δ 0.9-1.00 (m, 1OH), 1.38-1.50(m, 14H), 1.6-1.8 (m, 10H), 2.70 (dd, 1H),2.75 (dd, 1H), 2.76-2.98 (m, 2H), 2.72-3.04 (m, 4H), 3.09-3.18 (m, 1H), 3.32-3.42 (m, 1H), 3.5-3.9(m, 16H), 3.88-4.18 (m, 8H), 5 (d, 1H) 6.4 (s, 1H), 6.72-7.02

(m, 12H)

ESIMS, m/z for C₅₈H₈₀O₈N₄ [M+H]⁺: 961.292

### Example 79

### 2-[3-(3,4-Dimethoxy-phenyl)-5,6-dimethoxy-2-(4-methyl-piperazine-1-carbonyl)-indan-1-yl]-1-[4-2-ethoxy-phenyl)-piperazin-1-yl]-ethanone

### AV-256

Prepared analogously to example 74 from 1-(3,4-Dimethoxy-phenyl)-3-{2-[4-(2-ethoxy-phenyl)-piperazin-1-yl]-2-oxo-ethyl}-5,6-dimethoxy-indan-2-carboxylic acid in a yield 18% (11 mg) of a white solid.

1H NMR (300 MHz, CDCl₃) δ 1.2 (t, 3H), 2.18-2.4(m, 4H), 2.22 (s, 1H), 2.50 (dd, 1H),2.75 (dd, 1H), 2.8-2.9 (m, 1H), 3 (m, 4H), 3.3-3.6 (m, 9H), 3.7 (s, 3H), 3.8(s, 3H), 3.82 (s, 3H), 3.85 (s, 3H), 4.00-4.10(m, 3H), 4.95 (d, 1H) 6.4 (s, 1H), 6.72-7.02 (m, 8 H)

ESIMS, m/z for C₃₉H₅₀O₇N₄ [M+H]⁺: 687.4

### Example 80

### 1-(3,4-Dimethoxy-phenyl)-3-{2-[4-(2-ethoxy-phenyl)-piperazin-1-yl]-2-oxo-ethyl}-5,6-dimethoxy-indan-2-carboxylic acid dimethylamide

### AV-257

Prepared analogously to example 74 from 1-(3,4-Dimethoxy-phenyl)-3-{2-[4-(2-ethoxy-phenyl)-piperazin-1-yl]-2-oxo-ethyl}-5,6-dimethoxy-indan-2-carboxylic acid in a yield 10% (5.1 mg) of a white solid.

1H NMR (300 MHz, CDCl₃) δ 1.2 (t, 3H), 2.50 (dd, 1H),2.75 (dd, 1H), 2.8-2.85 (m, 1H), 2.82 (s, 3H), 3.01 (s, 3H), 3.0-3.10(m, 4H), 3.4-3.42 (m, 1H), 3.75-3.82 (m, 2H) 3.65 (s, 3H), 3.8(s, 3H), 3.82 (s, 3H), 3.85 (s, 3H), 4.00-4.20(m, 3H), 4.95 (d, 1H) 6.4 (s, 1H), 6.72-7.02 (m, 8H)

ESIMS, m/z for C₃₆H₄₅O₇N₃ [M+H]⁺: 632.5

### Example 81

### 2-{7-benzo[1,3]dioxol-5-yl-6-[4-(2-ethoxy-phenyl)-piperazin-1-carbonyl]-6,7-dihydro-5H-indeno[5,6-d][1,3]dioxol-5-yl}-1-[4-(2-ethoxy-phenyl)-piperazin-1-yl]-ethanone

### AV-258

Prepared analogously to example 74 from 5-Benzo[1,3]dioxol-5-yl-7-carboxymethyl-6,7-dihydro-5H-indeno[5,6-d][1,3]dioxol-6-carboxylic acid gave 2 isomers.

Isomer 1: White powder Y: 10% (25 mg) ESIMS, m/z for C₄₄H₄₈O₈N₄ [M+H]⁺: 761.4

1H NMR (300 MHz, CDCl₃) δ 1.4 (t, 6H), 2.50(m, 2H), 2.7 (m, 2H), 2.8-3.2 (m, 8H),3.4-3.9 (m, 10H), 3.92-4.2 (m, 6H), 4.95 (d, 1H) 5.8-7.02 (m, 13H)

Isomer 2: White powder Y: 10% (25 mg)

ESIMS, m/z for C₄₄H₄₈O₈N₄ [M+H]⁺: 761.4

1H NMR (300 MHz, CDCl₃) δ 1.4 (t, 6H), 2.20(m, 1H), 2.4 (m, 1H), 2.8-3.4 (m, 11H), 3.5-3.8 (m, 7H), 3.92-4.18 (m, 7H), 4.25 (d, 1H) 5.8-7.02 (m, 13H)

### Example 82

### N-{2-[(2-N-methyl)-4-tert-butyl-benzamide-3(3,4-dimethoxy-phenyl)-5,6-dimethoxy-indan-1-yl}-ethyl-4-tert-butyl-benzamide

### AV-259

A solution of 2-[2-aminomethyl-3-(3,4-dimethoxy-phenyl)-5,6-dimethoxy-indan-1-yl]-ethylamine (0.026 g, 0.06 mmol), in 4 ml anhydrous dichloromethane was prepared. To this solution was added morpholinomethyl polystyrene resin (0.2 g. reference Novobiochem 3.50 mmol/g). 4-tert-butyl-benzoyl chloride (0.070 g, 0.35 mmol) was added to the suspension. The mixture was shacked at room temperature overnight. The polystyrene resin was then filtered. And washed with dichloromethane. To the dichloromethane solution the polyamine resin the tris(2-aminoethylamine), (200 mg from Novabiochem). The suspension was shaken at room temperature overnight, then the mixture was filtered. The dichloromethane was evaporated under vacuum gave white powder. Fast filtration through small silica column with ethyl acetate gave 10 mg of white powder. Y: 14%ESIMS, m/z for C₄₄H₅₄O₆N₂ [M+Na]+; 729.3

### Example 83

### N-{2-[(2-N-methyl)-4-trifluoromethoxy-benzamide-3(3,4-dimethoxy-phenyl)-5,6-dimethoxy-indan-1-yl}-ethyl-4-trifluoromethoxy-benzamide

### AV-260

Prepared analogously to example 82 from 4-(trifluoromethoxy)benzoyl chloride in a 5 mg of white powder.

Y: 6% ESIMS, m/z for C₃₈H₃₆F₆O₈N₂ [M+Na]+: 785.2

### Example 84

### N-{2-[(2-N-methyl)-4-propyl-benzamide-3(3,4-dimethoxy-phenyl)-5,6-dimethoxy-indan-1-yl}-ethyl-4-propyl-benzamide

### AV-262

Prepared analogously to example 83 from 4-butyl-benzoyl chloride in 10 mg of white powder.

Y: 14% ESIMS, m/z for C₄₂H₅₀O₆N₂ [M+Na]+: 701.5

### Example 85

### N-{2-[(2-N-methyl)-4-bromo-benzamide-3(3,4-dimethoxy-phenyl)-5,6-dimethoxy-indan-1-yl}-ethyl-4-bromo-benzamide

### AV-263

Prepared analogously to example 83 from 4-bromo-benzoyl chloride in 5 mg of a white powder.

Y: 6%, ESIMS, m/z for C₃₆H₃₆O₆N₂Br₂ [M+2Na]+: 796.5, [M+Na]⁺: 773.0, M:752.0

### Example 86

### N-{2-[(2-N-methyl)-benzamide-3(3,4-dimethoxy-phenyl)-5,6-dimethoxy-indan-1-yl}-ethyl-benzamide

### AV-264

Prepared analogously to example 83 from benzoyl chloride in 12 mg of a white powder.

Y: 20%, ESIMS, m/z for C₃₆H₃₈O₆N₂: 594.5

### Example 87

N{2-[(2-N-methyl)-2-methoxy-benzamide-3(3,4-dimethoxy-phenyl)-5,6-dimethoxy-indan-1-yl}-ethyl-2-methoxy-benzamide

### AV-265

Prepared analogously to example 83 from orto-anisoyl chloride in 5 mg of a white powder.

Y: 7.6% ESIMS, m/z for C₃₈H₄₂O₈N₂ [M+Na]⁺: 678.3, [M+H]: 655.2

### Example 88

### Compounds of Formula I Inhibit P-gp as Evidenced by Cytotoxicity Assays

Cytotoxicity assays were performed with many of the compounds of Formula I to verify their usefulness as inhibitors of P-gp.

Parental NIH3T3 Swiss mouse embryo cell line was obtained from American Type Culture Collection and was grown in Dulbecco's Modified Eagles Medium supplemented with 4.5 g/L glucose, 10% fetal bovine serum, 2 mM L-glutamine, and 0.01 mg/ml gentamicin. Drug resistant NIH3T3 cells were derived by transfection of the human MDR₁ cDNA into parental NIH3T3 cells and were maintained in similar medium supplemented with 60 ng/ml of colchicine. As mentioned above, the human MDR₁ gene encodes the drug transporting membrane protein P-glycoprotein. The human ileocecal adenocarcinoma cell line HCT-8 was grown in RPMI-1640 medium supplemented with 10% horse serum, 1 mM sodium pyruvate and 0.01 mg/ml gentamicin. All cells were maintained in a humidified atmosphere with 5% CO₂ at 37° C.

Parental and MDR₁-expressing NIH 3T3 cells were plated at a density of 2.5-3.0×10³ cells/well in 96-well microtiter plates and were exposed to 50 nM of doxoriubicin, 7.5 nM vinblastine, 75 nM colchicine or 300 nM paclitaxel for 72 hours. Cell viability was determined with the colorimetric MTT (3-(4,5-dimethylthiazol-2-yl)-2,5-diphenyl tetrazolium) assay as previously described (Mosmann T., J. Immunol. Methods, 65: 55-63 (1983); Hansen M. B. et al., J. Immunol. Methods, 119: 203-210 (1989)) and the absorbance was measured at 570 nm.

The efficacy of particular compounds of Formula I, in a 5 μM amount, on the modulation of cytotoxicity of the doxorubicin is shown in Table I. The data are presented as a percentage increase in doxorubicin cytotoxicity in the presence of the compounds versus in their absence. Similar results evidencing the utility of the compounds of the invention for increasing sensitivity of multidrug resistant cells were obtained when the assays were repeated with taxol, vinblastive, and colchicine.

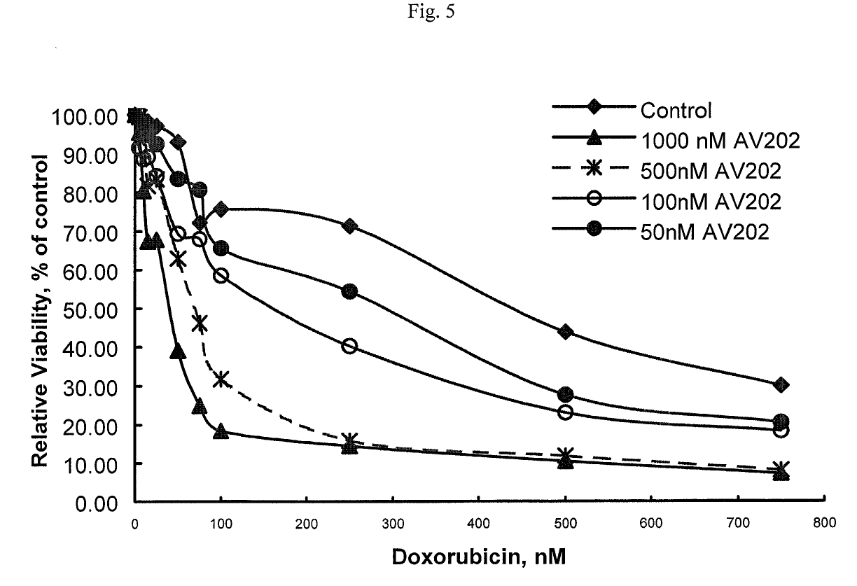

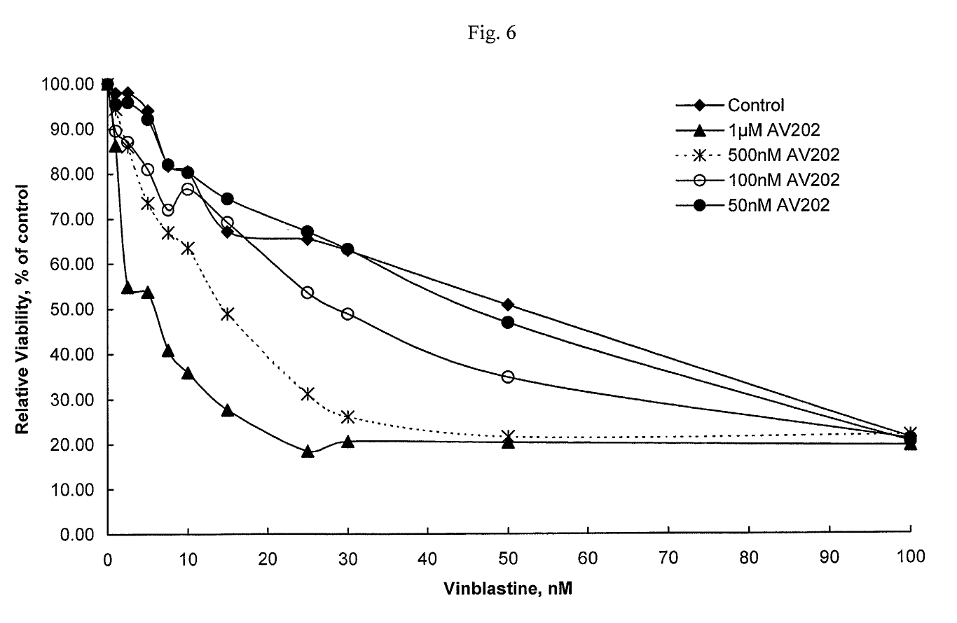

FIG. 5 depicts an example of the dose responsive sensitization of multidrug resistant cells to doxorubicin, 0-750 nM, in the presence of 50-1000 nM of one of the compounds, AV202, of Formula I. Similarly, FIG. 6 depicts dose response data inhibition of P-glycoprotein in the presence of 50-1000 nM AV202 in the presence of 0-100 nM vinblastine. Similar dose response data were obtained with 0-2000 nM coichicine and 0-3000 nM vinblastine (data not shown). Dose response studies for seven other compounds of the invention, AV-171, AV197, AV-198, AV-206 and AV-207, also gave similar results (data not shown).

As evidenced from these data, the compounds of Formula I are highly useful as P-gp inhibitors, and particularly in the multidrug resistance context.

TABLE I

| | Modulation of | % Inhibition of R123 |
|---|---|---|
| Compound | Drug Resistance | Transport |
| AV-159 | 56.06 | 81.4 |
| AV-161 | 75.91 | 64.1 |
| AV-162 | 65.83 | 83.1 |
| AV-163 | 72.55 | 89.7 |
| AV-164 | 70.03 | 85.1 |
| AV-165 | 6.45 | 14.6 |
| AV-166 | 9.67 | −143.0 |
| AV-167 | 68.21 | 66.3 |
| AV-168 | 73.81 | 81.5 |
| AV-169 | 71.29 | 77.1 |
| AV-170 | 61.07 | 62.7 |
| AV-171 | 66.11 | 92.9 |
| AV-172 | 68.57 | 86.7 |
| AV-173 | 73.23 | 80.2 |
| AV-174 | 18.50 | 23.1 |
| AV-175 | 52.50 | 71.3 |
| AV-176 | 8.65 | 11.5 |
| AV-177 | 40.32 | 9.4 |
| AV-178 | 54.50 | 79.0 |
| AV-179 | 58.07 | 87.7 |
| AV-180 | 42.84 | 40.4 |
| AV-181 | 6.13 | 6.0 |
| AV-182 | 23.87 | 25.0 |
| AV-183 | 52.24 | 63.0 |
| AV-184 | 50.58 | 53.7 |
| AV-185 | 18.48 | 25.1 |
| AV-186 | 64.87 | 65.8 |
| AV-187 | 23.50 | 2.7 |
| AV-188 | 75.39 | 87.6 |
| AV-189 | 64.78 | 60.0 |
| AV-190 | 65.35 | 52.2 |
| AV-191 | 63.49 | 64.9 |
| AV-192 | 19.61 | 20.2 |
| AV-193 | 25.20 | 11.8 |
| AV-194 | 64.62 | 86.9 |
| AV-195 | 60.82 | 76.8 |
| AV-196 | 41.47 | 64.4 |
| AV-197 | 73.65 | 88.9 |
| AV-198 | 58.65 | 84.7 |
| AV-199 | 61.63 | 84.0 |
| AV-200 | 61.40 | 86.4 |
| AV-201 | 66.44 | 83.8 |
| AV-202 | 59.68 | 88.4 |
| AV-203 | 84.65 | 77.9 |
| AV-204 | 64.60 | |
| AV-205 | 35.56 | 53.60 |
| AV-206 | 54.78 | 88.22 |
| AV-207 | 55.08 | 89.70 |
| AV-208 | 51.78 | 86.08 |
| AV-209 | 1.16 | 47.30 |
| AV-223 | 36.55 | 74.50 |
| AV-234 | 12.50 | 37.58 |
| AV-239-1 | 55.3 | 90.10 |
| AV-239-2 | 60 | 91.38 |
| AV-250 | −7.1 | 55.88 |
| AV-251-1 | −6.9 | 79.26 |
| AV-251-2 | 2.4 | 75.44 |
| AV-252 | −0.5 | 64.17 |
| AV-253 | 19.8 | 92.03 |
| AV-254 | 4.1 | 18.70 |
| AV-255 | 6.7 | 96.0 |
| AV-256 | 8.7 | 75.61 |
| AV-257 | 8.7 | 80.42 |
| AV-258-1 | 7.9 | 69.87 |
| AV-258-2 | 19.2 | 67.09 |
| AV-259 | 3.9 | 84.87 |
| AV-260 | 2.1 | 73.84 |
| AV-262 | 18.0 | 83.47 |
| AV-263 | 12.2 | 80.78 |
| AV-264 | 6.3 | 83.51 |
| AV-265 | −0.2 | 93.0 |

### Example 89

### Compounds of Formula I Inhibit P-gp as Evidenced by the Rhodamine Transport Assay

Rhodamine 123 transport was examined as previously described (Hunter J. et al., Simmons, Br. J. Cancer, 64: 437444 (1991); Kim A. E. et al., J. Pharm. Exp. Ther., 286: 1439-1445 (1998)) using HCT-8 cells. Particularly, cells were grown in 6 well Corning Transwell dishes until a tight monolayer was formed. Rh123 was added at a final concentration of 15 μM to the basal or apical compartments and 200 μl samples were taken at the indicated times from the opposite chamber. Fluorescence of Rh123 in the media samples was measured using a fluorescence plate reader with an excitation wavelength of 485 nm and an emission wavelength of 530 nm. Rh123 is a well-established substrate for P-glycoprotein. This assay demonstrates the ability of the compounds described herein to modulate the P-glycoprotein mediated transport. Table I, presented above in conjunction with Example 46 demonstrates the activity of many of the compounds of Formula I, in a 10 μM amount, to potentiate P-glycoprotein mediated transport of Rhodamine 123. Therefore, these compounds would be useful as P-gp inhibitors in patient use.

All publications and patent applications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the appended claims.

What is claimed is:

1. A compound of the formula: 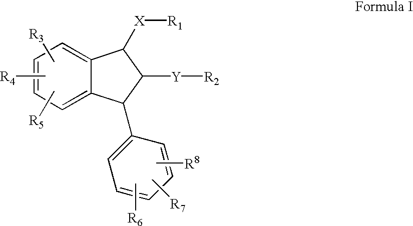
wherein:
R₁ and R₂ are each independently —OR₉ or —NR₁₀R_{11;}
R₃, R₄, R₅, R₆, R₇, and R₈ are each independently hydrogen, C₁₋₁₀ alkyl, C₂₋₁₀ alkenyl, C₂₋₁₀ alkynyl, C₁₁O alkoxy, phenyl, phenoxy, benzyl, benzyloxy, C₃₋₈ cycloalkyl, N(R₁₂)₂, NHCOR₁₃, S(O)_{q}C₁₋₁₀ alkyl, OH, or halogen; wherein C₁₋₁₀ alkyl, C₂₋₁₀ alkenyl, or C₂₋₁₀ alkynyl may be optionally substituted by COOH, OH, CO(CH₂)ₙCH₃, CO(CH₂)ₙ, CH₂N(R₁₂)₂, or halogen; or R₃ and R₄, R₄ and R₅, R₆ and R₇, or R₇ and R₈ together may be —O—A—O— on contiguous carbons;
R_{g} is C₁₋₁₀ alkylene, C₂₋₁₀ alkenylene, C₂₋₁₀ alkylidene, or C₂₋₁₀ alkynylene, all of which may be linear or branched or phenylene, all of which may be unsubstituted or substituted by one or more OH, COOH, alkoxy, NHR₁₂, N(R₁₂)₂, NHCOR₁₃ or halogen; or R_{g} is alkylsilyl, arylsilyl or alkylarylsilyl;
R₁₀ and R₁₁ are each independently C₁₋₁₀ alkylene, C₁₋₁₀ alkenylene, C₂₋₁₀ alkylidene, C₂₋₁₀ alkynylene, S(O)_{q}(R₁₄), C(O)NH(R₁₄), and C(O)_{q}(R₁₄), all of which may be linear or branched, phenylene or benzylene, all of which may be unsubstituted or substituted by one or more OH, COOH, alkoxy, NHR₁₂, N(R₁₂)₂, NHCOR₁₃ or selected from the following group: 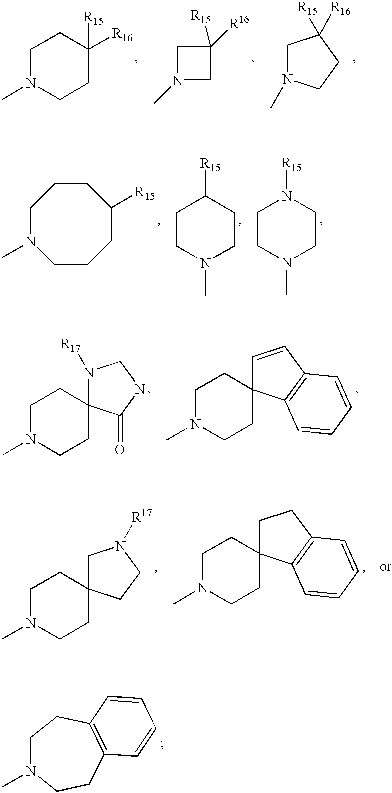
R₁₂ is hydrogen, C₁₋₁₀ alkyl, C₂₋₁₀ alkenyl, benzyl, or aryl, all of which may be unsubstituted or substituted by one or more OH, COOH, NH₂, secondary amine, tertiary amine, tetrazole, or PO₃H_{2;}
R₁₃ is C₁₋₁₀ alkyl, C₂₋₁₀ alkenyl, benzyl, or aryl, all of which may be unsubstituted or substituted by one or more OH, COOH, NH₂, secondary amine, tertiary amine, tetrazole, or PO₃H₂;
R₁₄ is C₁₋₁₀ alkyl, C₂₋₁₀ alkenyl, C₂₋₁₀ alkynyl, C₃₋₆ cycloalkyl, phenyl, and benzyl; wherein C₁₋₁₀ alkyl, C₂₋₁₀ alkenyl, C₂₋₁₀ alkynyl maybe optionally substituted by COOH, CO(CH₂)ₙCH₃ or OH;
R₁₅ and R₁₆ are each independently hydrogen, aryl, C₁₋₁₀ alkyl, C₂₋₁₀ alkenyl, and C₂₋₁₀ alkynyl, all of which may be unsubstituted or substituted by one or more CH₂OH, N(R₁₂)₂, NHCOR₁₃, OH, or halogen; wherein aryl is naphthyl, indolyl, pyridyl, thienyl, oxazolidinyl, oxazolyl, thiazolyl, isothiazolyl, pyrazolyl, triazolyl, tetrazolyl, imidazolyl, imidazolidinyl, thiazolidinyl, isoxazolyl, oxadiazolyl, thiadiazolyl, morpholinyl, piperidinyl, piperazinyl, pyrrolyl or pyrimidyl, all of which may be unsubstituted or substituted by one or more R₁₇, R₁₈, R₁₉; wherein if R₁₅ occurs without R₁₆, R₁₅ is not hydrogen;
R₁₇, R₁₈, R₁₉ are each independently hydrogen, C₁₋₁₀ alkyl, C₂₋₁₀ alkenyl, C₂₋₁₀ alkynyl, phenyl, benzyl, C₃₋₈ cycloalkyl, C₁₋₁₀alkoxy, S(O)_{q}C₁₋₁₀ alkyl, N(R₁₄)₂, NHCOR₆, OH, or halogen; wherein C₁₋₁₀ alkyl, C₂₋₁₀ alkenyl or C₂₋₁₀ alkynyl may be optionally substituted by COOH, CO(CH₂)ₙCH₃, CO(CH₂)ₙ, CH₂N(R₁₄)₂, OH or halogen;
X and Y are each independently —CH₂—, —(CH₂)2—, —(CH₂)₃—, C═O, CH₂C(O), (CH₂)₂C(O), CH₂SO₂, and (CH₂)₂SO₂;
q is zero, one, two, or three;
n is an integer from zero to six;
or a diastereomer, enantiomer or pharmaceutically acceptable salts thereof.

2. The compound of claim 1 wherein X is CH₂(0), and Y is C═O.

3. The compound of claim 2 wherein R₃ and R₆ are H; and R₄, R₅, R₇ and R₈ are —OCH₃.

4. The compound of claim 3 which is 2-[2-(4-benzylpiperidine-1-carbonyl)-3-(3,4-dimethoxy-phenyl)-5,6-dimethoxy-indan-1-yl]-1-(4-benzylpiperidin-1-yl)-ethanone.

5. The compound of claim 3 which is 2-{3-(3,4-dimethoxyphenyl)-5,6-dimethoxy-2-[4-(2-ethoxyphenyl)-piperazin-1-carbonyl]-indan-1-yl}-1-[4-(2-ethoxyphenyl)-1-yl]-ethanone.

6. The compound of claim 1 wherein X is —(CH₂)₂—, and Y is —CH₂—.

7. The compound of claim 6 wherein R₃ and R₆ are H; and R₄, R₅, R₇ and R₈ are -OCH₃.

8. The compound of claim 1 wherein X is —(CH₂)₂—, and Y is —CH₂—; R₂ is —OCH₃; R₃ and R₆ are H; and R₄, R₅, R₇ and R₈ are —OCH₃.

9. The compound of claim 8 which is 1-benzyl-4-{2-[3-(3,4-dimethoxyphenyl)-5,6-dimethoxy-2-methoxymethyl-indan-1-yl]-ethyl}-piperazine.

10. A method of inhibiting P-glycoprotein-mediated transport, the method comprising administering a compound of Formula I.

11. The method of claim 10 wherein the compound of Formula I is selected from the group presented in Table I.

12. The method of claim 11 wherein the compound has a percentage inhibition of Rhodamine 123 transport of at least 30%.

13. The method of claim 12 wherein the compound has a percentage inhibition of Rhodamine 123 transport of at least 50%.

14. The method of claim 13 wherein the compound has a percentage inhibition of Rhodamine 123 transport of at least 80%.

15. The method of claim 11 wherein the compound of Formula I is coadministered with a pharmaceutical compound.

16. The method of claim 15 wherein the coadministered pharmaceutical compound has a percentage increase in cytotoxicity value of at least 30%.

17. The method of claim 16 wherein the coadministered pharmaceutical compound has a percentage increase in cytotoxicity value of at least 50%.

18. The method of claim 17 wherein the coadministered pharmaceutical compound has a percentage increase in cytotoxicity value of at least 80%.

19. Use of a compound of Formula I as an inhibitor of P-glycoprotein-mediated transport.

20. An inhibitor of P-glycoprotein-mediated transport comprising a compound of Formula I.

21. A method of increasing bioavailability of an orally administered pharmaceutical compound, the method comprising:
orally coadministering (1) the pharmaceutical compound to a mammal in need of treatment with the pharmaceutical compound and (2) a compound of Formula I in an amount of the compound of Formula I sufficient to provide bioavailability of the pharmaceutical compound in the presence of the compound of Formula I greater than bioavailability of the pharmaceutical compound in the absence of the compound of Formula I.

22. A method of treating multidrug resistance in mammals, the method comprising administering to a mammal in need of such treatment a therapeutically effective amount of a compound of Formula I.

23. A composition for inhibition of P-glycoprotein-mediated transport in mammals, the composition comprising a compound of Formula I in a pharmaceutically acceptable carrier.

24. A method of treating a tumor, the method comprising coadministering to a mammal in need thereof a therapeutically effective amount of a compound of Formula I and a therapeutic agent.

25. The method of claim 23 wherein the tumor is drug-resistant.

26. The method of claim 23 wherein the tumor has converted from drug-sensitive to drug-resistant.

27. The method of claim 23 wherein the therapeutic agent is a P-glycoprotein substrate.

28. The method of claim 23 is selected from doxorubicin, vinblastine, vincristine, epipodophyllotoxin, taxanes, paclitaxel, docetaxel, etoposide, tenopiside, colchicines, daunorubicin, topotecan, actinomycin D, mitoxantrone, mitomycin C.

29. A method of preventing multidrug resistance in tumor cells, the method comprising administering an effective amount of a compound of Formula I to the tumor cells.

30. A method of treating a tumor, the method comprising administering to a mammal in need thereof a therapeutically effective amount of a composition, the composition including a therapeutic agent and a compound of Formula I.

31. A method for increasing the sensitivity of tumor cells that have converted from sensitivity to therapeutic agents to resistance to the therapeutic agents, the method comprising coadministering to a mammal a therapeutically effective amount of a compound of Formula I and at least one of the therapeutic agents.

32. A pharmaceutical composition for increasing the sensitivity of tumor cells that have converted from sensitivity to therapeutic agents to resistance to the therapeutic agents, the pharmaceutical composition comprising (1) a therapeutically effective amount of a compound of Formula I, (2) at least one of the therapeutic agents, and (3) a pharmaceutically acceptable carrier.

33. A method of converting a non-orally bioavailable pharmaceutical composition into an orally bioavailable pharmaceutical composition, the method comprising formulating a composition including a compound of Formula I and the non-orally bioavailable pharmaceutical composition and optionally a pharmaceutically acceptable carrier.

34. An orally bioavailable pharmaceutical composition produced by the method of claim 33.

35. A method of delivering a pharmaceutical compound to the central nervous system of a patient, the method comprising coadministering the pharmaceutical compound with a compound of Formula I.
